(19) United States
(12) Patent Application Publication
(10) Pub. No.: US 2022/0322993 A1
(43) Pub. Date: Oct. 13, 2022

(54) Neurofeedback Training to Promote Sharp Wave Ripples

(71) Applicant: The Regents of the University of California, Oakland, CA (US)

(72) Inventors: Loren M. Frank, San Anselmo, CA (US); Anna Gillespie, San Francisco, CA (US)

(21) Appl. No.: 17/633,749

(22) PCT Filed: Aug. 13, 2020
(86) PCT No.: PCT/US20/46216

§ 371 Date: Feb. 8, 2022

Related U.S. Application Data
(60) Provisional application No. 62/887,875, filed on Aug. 16, 2019.

(51) Int. Cl. A61B 5/375; A61B 5/245; A61B 5/055; A61B 5/291; A61B 5/297; A61B 5/374; A61N 1/36

(52) U.S. Cl. CPC A61B 5/375; A61B 5/245; A61B 5/055; A61B 5/291; A61B 5/297; A61B 5/374; A61N 1/36082

(57) ABSTRACT

Provided are methods and systems of for enhancing or increasing memory performance and/or memory retrieval in a subject using neurofeedback training. Also provided herein are methods and systems for modulating hippocampal replay in a subject using neurofeedback training. Also provided herein are methods and systems for modulating sharp wave ripple (SWR) activity in a subject using neurofeedback training

### CROSS-REFERENCE TO RELATED

This application claims the benefit of U.S. Provisional Patent Application No. 62/887,875, filed Aug. 16, 2019, which application is incorporated herein by reference in its entirety

### STATEMENT REGARDING FEDERALLY SPONSORED RESEARCH

This invention was made with government support under Grant No. MH105174 awarded by the National Institutes of Health. The government has certain rights in the invention.

### INTRODUCTION

As a primary symptom of normal aging and diseases of aging such as Alzheimer's disease, memory loss is a major cause of reduced quality of life and decreased independence for millions of adults worldwide. However, existing therapies for the treatment of memory loss provide only limited and temporary improvement of symptoms, and are only effective in a subset of the patient population. No therapies have been demonstrated to prevent the onset of memory loss. Memory is known to critically depend on the hippocampus, a highly conserved subcortical brain structure found in all mammalian species. Decades of research have established that the hippocampus in humans and in model organisms is critical for diverse aspects of memory, including but not limited to the initial encoding of experience, the storage and consolidation of the neural representation of experience, and the retrieval of past experience to guide future actions. Although many of the neural correlates of memory are unknown, one pattern of hippocampal activity has been reliably linked with several stages of memory processing. This phenomenon, termed hippocampal replay, refers to brief events during which the neural ensembles corresponding to prior experiences are reactivated in a time-compressed manner, recapitulating the neural representation of the original experiences (e.g. Wilson and McNaughton, (1994) Science, Vol. 265, pp. 676-679; Foster and Wilson, (2006) Nature, Vol. 440, pp. 680-683). Replay tends to occur during sharp wave ripples (SWRs), distinctive high frequency fluctuations in the hippocampal local field potential (Buzsaki (2015) Hippocampus, 25(10)). The detection of SWRs using hippocampal electrodes has become a common proxy for detecting replay events. Both hippocampal replay and SWRs have been observed in diverse species including mice, rats, bats, primates, and humans, and consistently related to memory functions, indicative of a highly conserved mechanism for memory processing. Furthermore, evidence from studies of aging and models of disease have demonstrated that abnormalities in replay and SWRs can precede and accompany cognitive decline. SWR manipulation studies in rodents have achieved bidirectional control of memory performance, providing strong causal evidence for SWRs in regulating memory abilities. Together, these findings motivate a therapeutic strategy based on promoting or enhancing SWRs and replay.

Existing technologies are not amenable to promoting or enhancing SWRs or replay in humans. There is a need for methods to improve or promote physiologically relevant SWRs in order to combat memory loss and/or to enhance memory ability.

### SUMMARY

Provided are methods and systems of for enhancing or increasing memory performance and/or memory retrieval in a subject using neurofeedback training. Also provided herein are methods and systems for modulating hippocampal replay in a subject using neurofeedback training. Also provided herein are methods and systems for modulating sharp wave ripple (SWR) activity in a subject using neurofeedback training.

### BRIEF DESCRIPTION OF THE DRAWINGS

The invention may be best understood from the following detailed description when read in conjunction with the accompanying drawings. Included in the drawings are the following figures:

FIG. 1 depicts a method of detecting SWRs in a rodent. A, Bilateral implant of 20-28 electrode bundles (tetrodes) targeting dorsal CA1 region of the hippocampus. Tetrodes are gradually lowered until they are located in the pyramidal cell layer. B, After recovery, data is acquired from an awake, freely moving rat. C, Data from each tetrode is sampled at 1500 Hz (raw LFP) and filtered for 150-250 Hz frequency activity. The mean and standard deviation of the envelope of the filtered signal is calculated during a period without SWRs. SWRs are detected as deviations exceeding 2 standard deviations above the mean on at least 2 out of 4 tetrodes simultaneously. The size of each SWR event (annotated below each sample event) is calculated post-hoc as the maximum standard deviation threshold at which the event would be detected.

FIG. 2 depicts the stages of the behavioral paradigm in which SWR-triggered neurofeedback is provided during the performance of a spatial memory task. Each port in the maze environment is equipped with an infrared photogate which detects the nose of the rodent subject in the port and can deliver a drop of evaporated milk to the subject as food reward. A, Each trial of the task is initiated when the rodent subject triggers the Home port and consumes the provided reward. Subsequently, one of the central ports will illuminate (randomly chosen each trial). B, The subject must visit the illuminated port and maintain nose position in the port until an auditory cue occurs. At the SWR port, the subject must maintain position in the port until a SWR is detected. Detection of the SWR will trigger the auditory cue and reward delivery. At the Control port, the subject must maintain position in the port for a delay period, irrespective of SWR occurrence. This delay is chosen to match the length of time taken to generate a SWR on recent previous SWR trials. C, After receiving the auditory cue and reward successful completion of either SWR or control port criteria, the subject must visit one of the eight outer arm ports. Only one of the eight outer arm ports will deliver reward such that the subject must explore various arms on each trial until the rewarded arm (goal) is discovered. The subject will continue to receive reward at the goal arm for subsequent trials. After 4-15 trials of goal arm visits, the goal arm will be reset to a new location and the subject must adapt its behavior to identify and receive reward at the new goal location. Each trial is ends when the subject returns to the Home port to initiate the next trial. Any deviations from this order of port visits triggers a 15-45 second period with no reward delivery from any port after which the subject must initiate a new trial at the Home port.

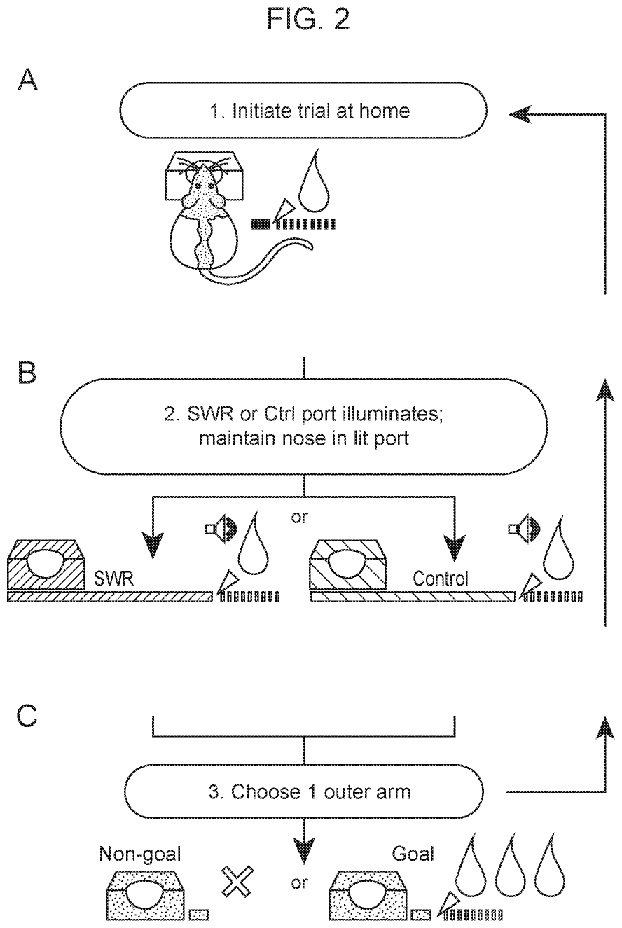

FIG. 3 depicts the spatial maze environment upon within which the rodent behavioral task is administered. Each rectangle denotes a reward Port. The environment is surrounded by 16 inch high walls and visual cues are provided outside the maze, on the walls of the room. The maze is located in a darkened, familiar room equipped with neural recording apparatus.

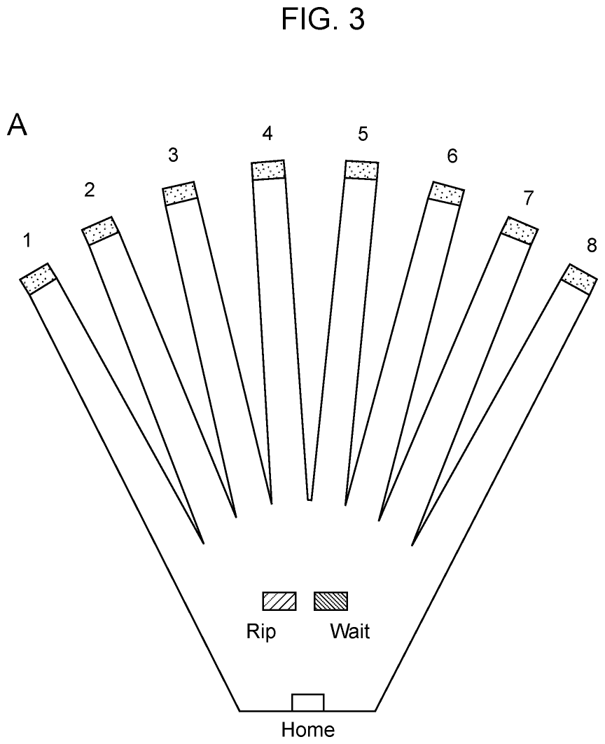

FIG. 4 depicts example trial data collected after the neurofeedback training has occurred. Raw and filtered LFP data from representative SWR (A) and Control (B) trials after training, beginning when the subject first triggers the port and ending when the subject leaves the port to continue the trial. SWR events are highlighted and annotated with their size (in standard deviations above mean). Red triangle indicates the time of auditory cue and reward delivery; dashed bars indicated reward consumption periods prior to departing from the Port. A greater number of SWR events occur preceding the trigger event in the SWR trial compared to the Control trial.

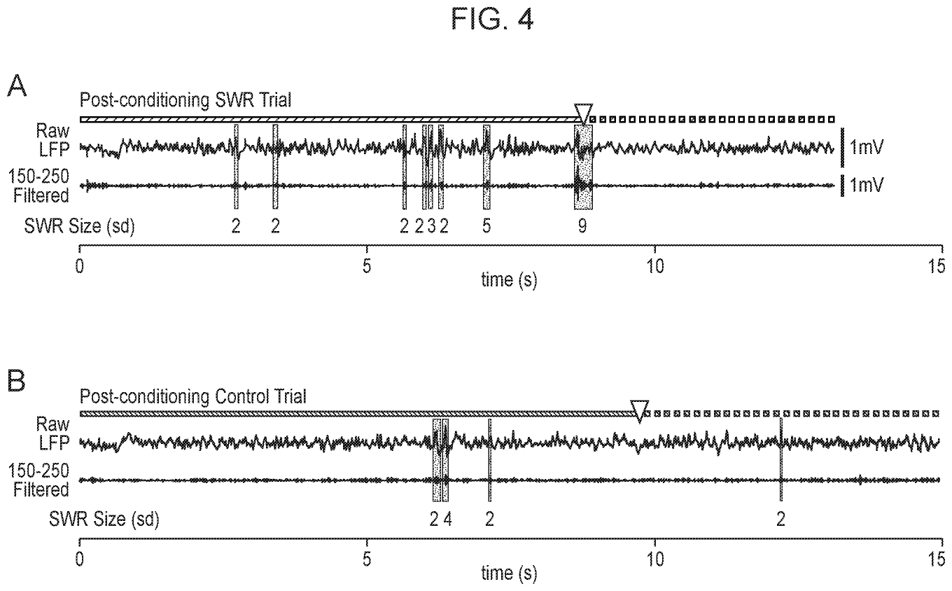

FIG. 5 depicts the gradual increase in SWR detection threshold across days of training. Subjects are required to generate increasingly large SWRs in order to satisfy criteria at the SWR port and continue with the task. In order to maintain performance, the subjects must learn to modulate SWR activity.

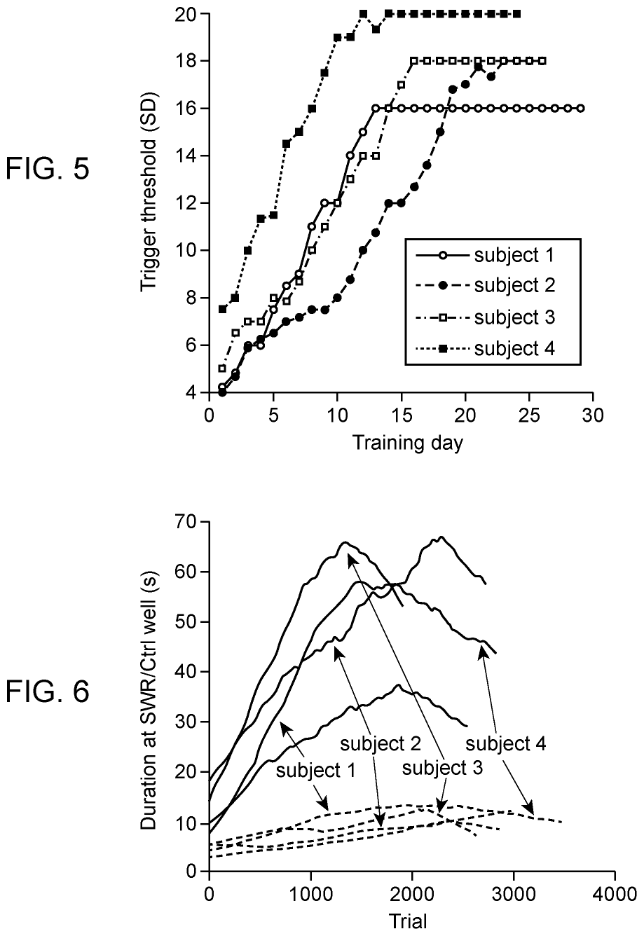

FIG. 6 depicts the observed (solid) and predicted (dashed) average length of time for SWR generation at the SWR port across training. Predicted values are based on the occurrence rate of SWRs greater or equal to the threshold size prior to training. The substantial difference between the predicted and observed durations demonstrates that the subjects have learn to modulate SWR processes rather than simply waiting for spontaneous large SWR events to occur.

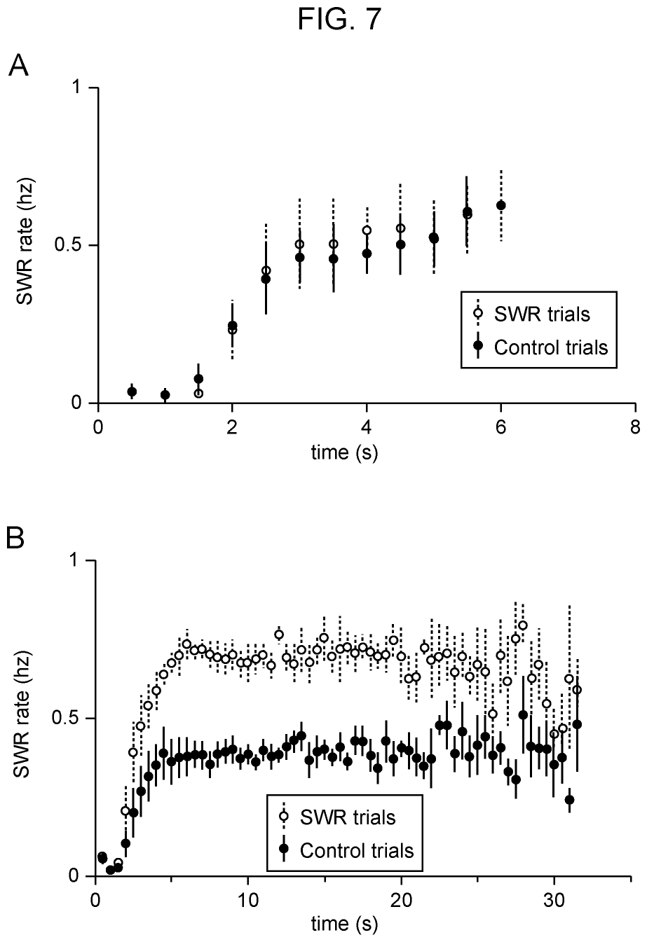

FIG. 7 depicts the SWR rate of occurrence calculated in 0.5s bins for the duration of time spent prior to cue and reward delivery at the SWR and Control ports before (A) and after (B) the neurofeedback training regime. After training, the SWR rate is substantially and consistently higher at the SWR port than at the Control port. p<0.0001 by permutation test; error bars are S.E.M. over n=4 subjects.

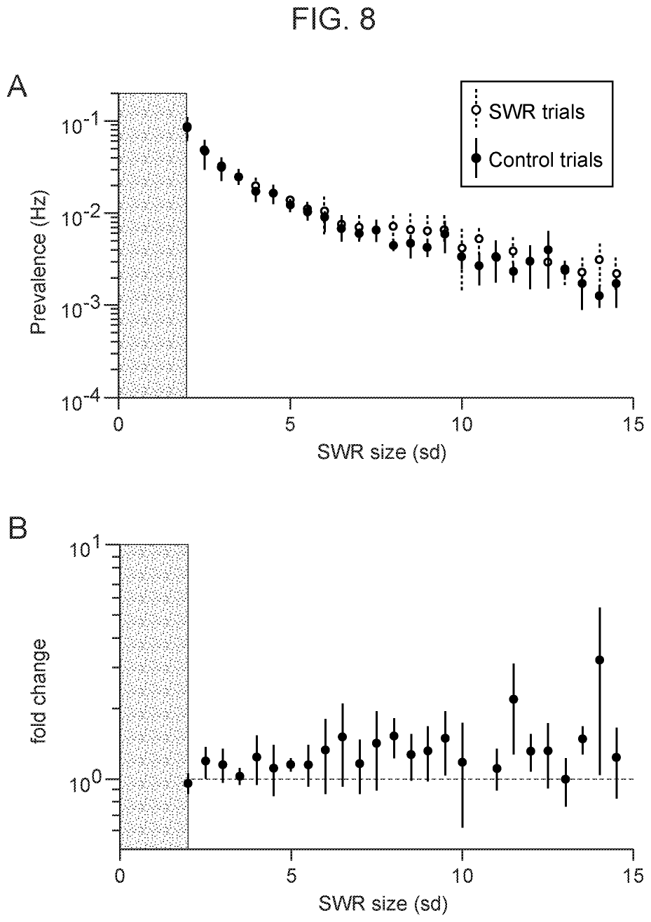

FIG. 8 depicts the prevalence (total events of each size detected per total time spent at SWR or Control port before cue; A) and the fold change of SWR prevalence relative to Control prevalence (B) before training and after training (C, D). Training causes a relatively uniform increase in SWR prevalence across all sizes of events. p<0.01 by permutation test; error bars are S.E.M. over n=4 subjects.

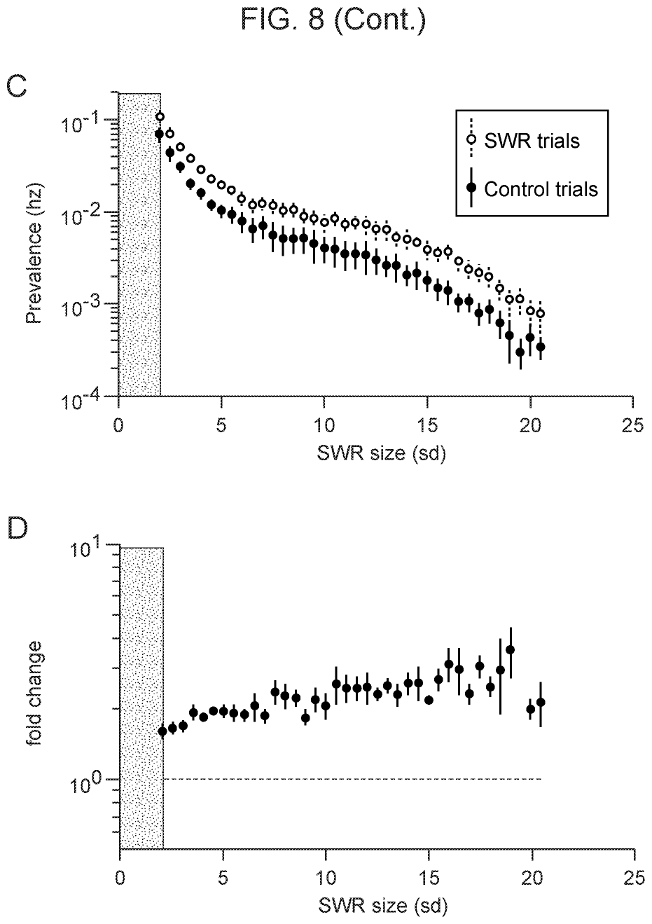

FIG. 9 depicts SWR occurrence rate (A), SWR prevalence (B), and fold change in SWR prevalence (C) after training with the triggering SWR event excluded from each trial. SWR rate and prevalence remain significantly higher, demonstrating that findings are not driven by the requisite presence of a single large SWR event (trigger) at the end of every SWR trial which is imposed by the structure of the behavioral paradigm. p<0.002 for A; p<0.002 for B by permutation test; error bars are S.E.M. over n=4 subjects.

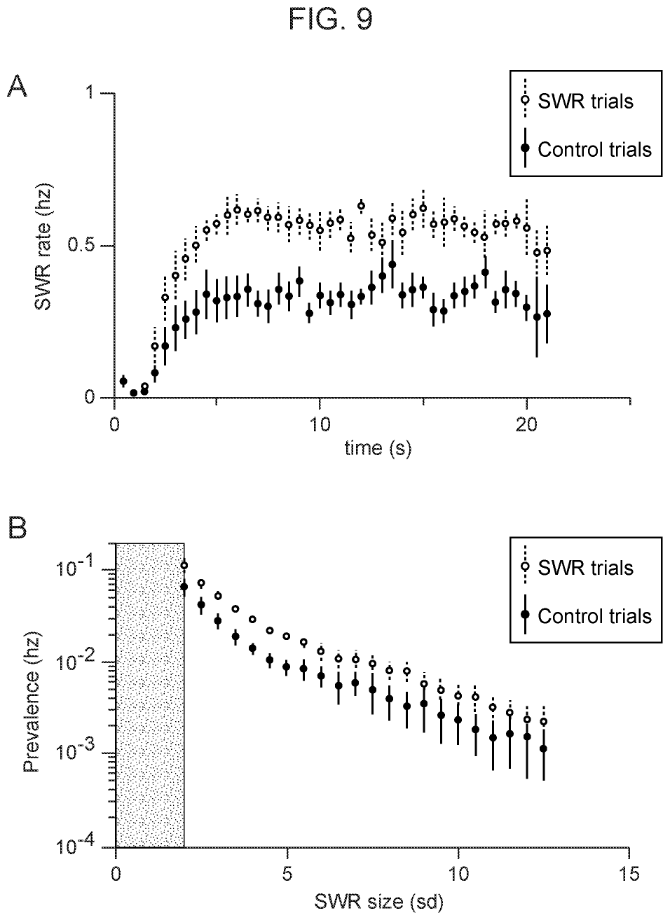

FIG. 10 depicts mean SWR size during the time preceding cue at SWR and Control ports. A includes the trigger SWR event in each SWR trial and shows a higher average SWR size during SWR trials compared to Control trials (p<0.0001 by t-test for each subject individually) while B excludes the trigger SWR from SWR trials and shows no difference in mean SWR size between the trial types. Comparison for each subject is shown.

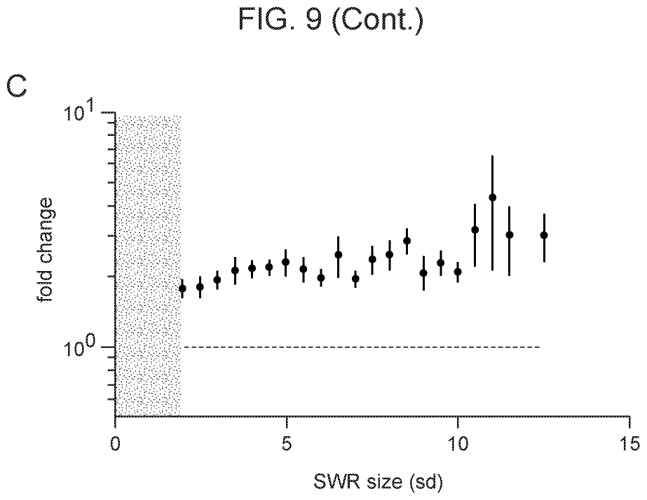

FIG. 11 depicts mean SWR length during the time preceding cue at SWR and Control ports. A includes the trigger SWR event in each SWR trial and shows a higher average SWR length during SWR trials compared to Control trials (p<0.0001 by t-test for each subject individually) while B excludes the trigger SWR from SWR trials and shows no difference in mean SWR length between the trial types. Comparison for each subject is shown.

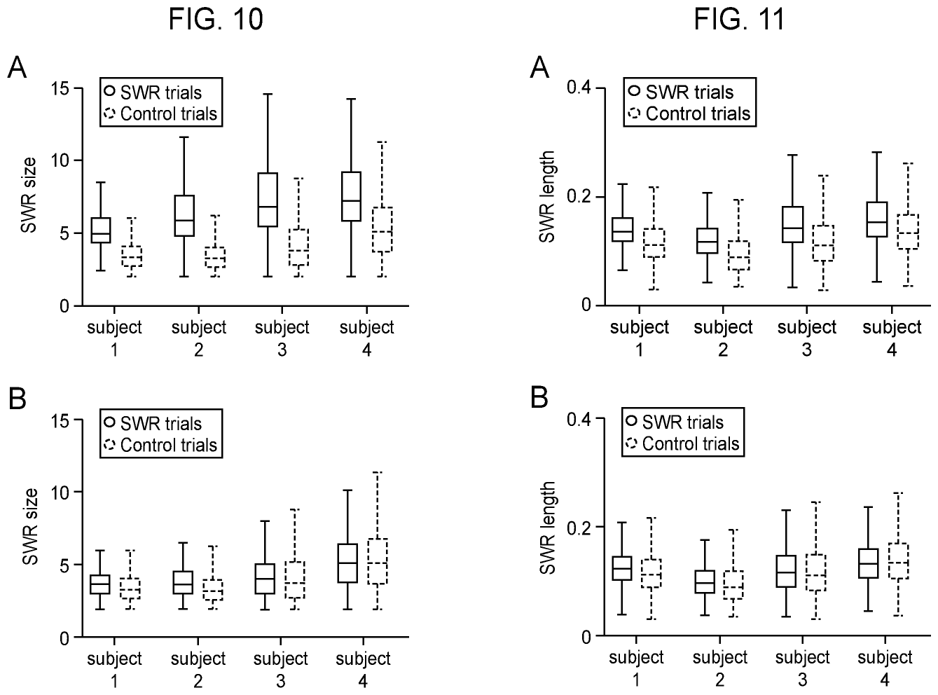

FIG. 12 depicts no difference in instantaneous frequency in the 150-250 Hz range during SWR events during the time preceding cue at SWR and Control ports. Comparison for each subject is shown.

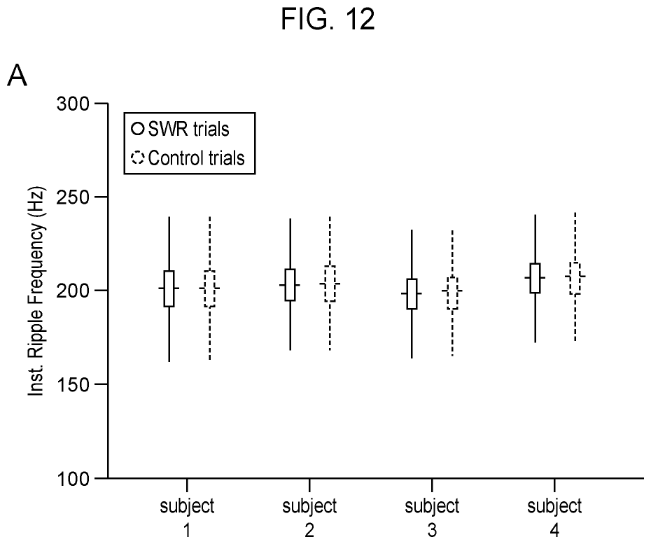

FIG. 13 depicts the SWR-triggered spectrogram for a representative CA1 pyramidal cell layer tetrode for SWR events during the time preceding cue at SWR (A) and Control ports (B). No difference in spectral features are evident.

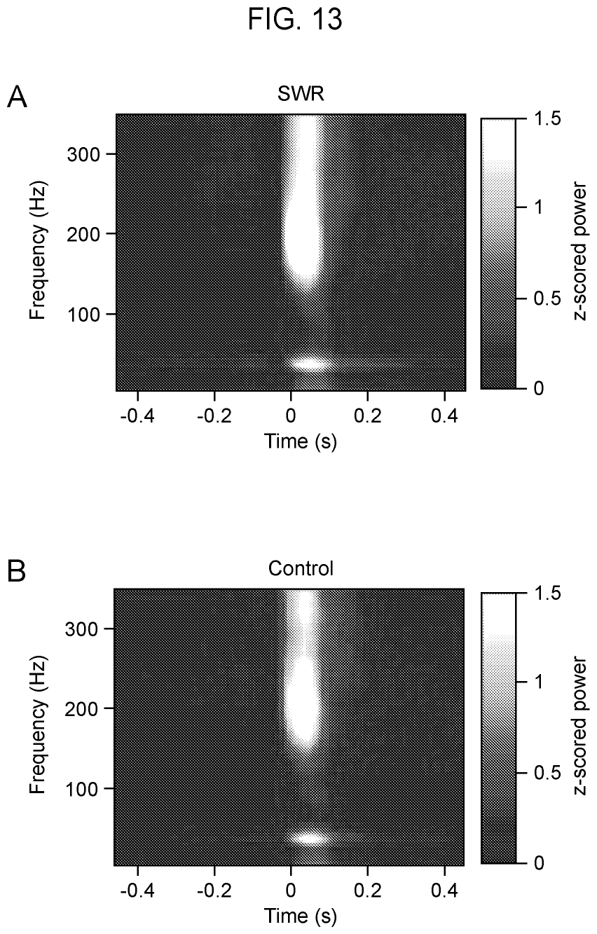

FIG. 14 depicts the classification of replay content types for SWR events during the time preceding cue at SWR and Control ports. Events can be described as one of seven categories: nonclassifiable (non), continuous (cont), fragmented (frag), hover (hov), compound continuous and fragmented (c+f), compound continuous and hover (c+h), compound fragmented and hover (f+h), compound including all three classifications (all). SWRs at SWR and Control ports do not differ in their content, suggesting that training increases SWRs with normal, physiological content and does not alter the general replay representations that occur.

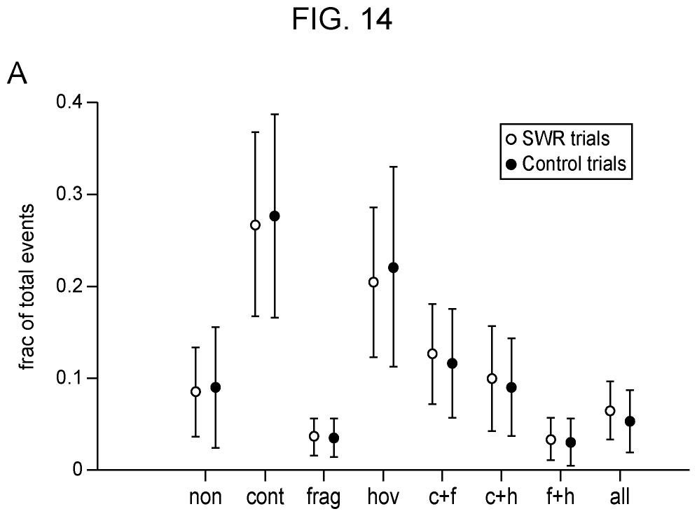

FIG. 15 depicts the SWR occurrence rate in 0.5s bins over the course of time at the home port (A), at the outer port on rewarded goal arm visits (B) before and after neurofeedback training. C, overall SWR rate during all quiet rest time in a sleep chamber after task performance before and after neurofeedback training. No difference in occurrence rates are observed, indicating that the effect of neurofeedback training is specific to the trial phase when it is required and does not alter SWR generation processes outside of that trial phase.

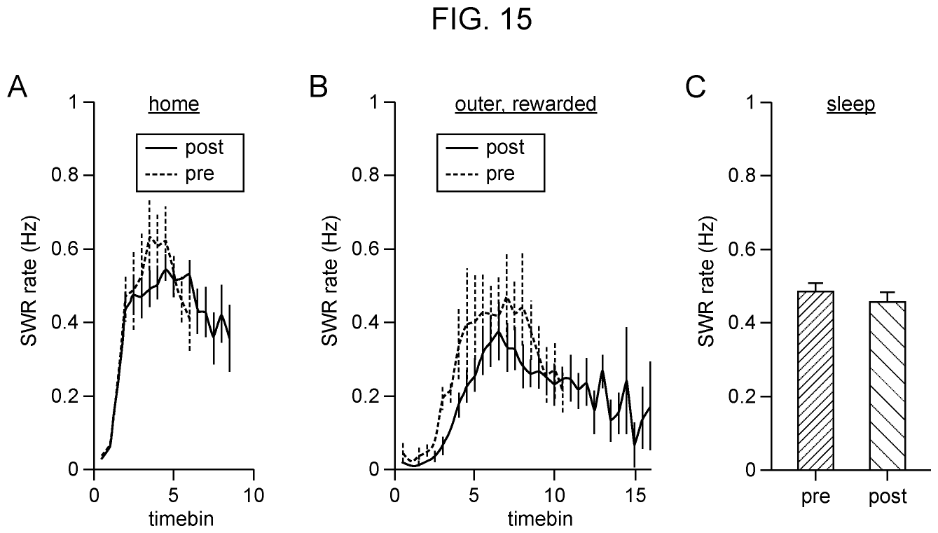

### DETAILED DESCRIPTION

Provided are methods and systems of for enhancing or increasing memory performance and/or memory retrieval in a subject using neurofeedback training. Also provided herein are methods and systems for modulating hippocampal replay in a subject using neurofeedback training. Also provided herein are methods and systems for modulating sharp wave ripple (SWR) activity in a subject using neurofeedback training. Systems and devices to enable neurofeedback triggered by SWR activity for implementing the above methods are also provided. Various steps and aspects of the methods will now be described in greater detail below.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” includes a plurality of such electrodes and reference to “the signal” includes reference to one or more signals, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. §112 are to be accorded full statutory equivalents under 35 U.S.C. § 112.

### Methods

Aspects of the present disclosure include methods of modulating hippocampal replay, SWR activity, enhancing or increasing memory performance, memory retrieval, and/or reducing memory loss, in a subject. In some embodiments, the method includes recording a plurality of signals from one or more regions of the brain of a subject. In some embodiments, the method includes filtering the plurality of signals to a frequency ranging from 20-250 Hz. In some embodiments, the method includes detecting SWR activity above a set threshold. In some embodiments, the method includes providing feedback to the subject triggered by the detection of SWR activity in the subject above a set threshold. In some embodiments, providing feedback to the subject modulates the SWR activity in the subject. In some embodiments, providing such feedback enhances or improves memory performance.

Neurofeedback training methods of the present disclosure are suitable for application to a variety of subjects. Subjects of interest include, but are not limited to mammals, both human and non-human, including the orders carnivore (e.g., dogs and cats), rodentia (e.g., mice, guinea pigs, and rats), lagomorpha (e.g. rabbits) and primates (e.g., humans, chimpanzees, and monkeys). The subject methods may be applied to human subjects of both genders and at any stage of development (i.e., neonates, infant, juvenile, adolescent, adult), where in certain embodiments the human subject is a juvenile, adolescent or adult. While the present invention may be applied to a human subject, it is to be understood that the subject methods may also be carried-out on other animal subjects such as, but not limited to, birds, mice, rats, dogs, bats, cats, livestock and horses. In some embodiments, the subject of the present systems is a healthy subject. In some embodiments, the subject of the present systems is a subject with a disease or disorder selected from the group consisting of: dementia, Alzheimer's disease, memory loss, epilepsy, and a combination thereof.

SWR activity, as used herein and in its conventional sense, refers to distinctive high frequency fluctuations in the hippocampal local field potential. In the rat, such fluctuations are brief (e.g. 80-200 ms), distinct patterns of 150-250 Hz oscillatory hippocampal network activity (e.g. Karlsson et al. Nat Neurosci, (2009) vol. 12, pp. 913-8). In humans, using commercially available macroelectrodes, SWR events are generally detected in a lower frequency range, namely, 80-140 Hz (see, e.g. Axmacher et al. (2008) Brain, vol. 131, pp. 1806-17, Vaz et al. (2019) Science, vol. 363, pp. 975-978). Hippocampal replay, as used herein and in its conventional sense, refers to brief events during which the ensemble of neurons corresponding to an experience is reactivated in a time-compressed manner. Such replay occurs during “offline” brain states, such as sleep or pauses in ongoing behavior, and is associated with SWR activity. During sleep, replay is critical for memory consolidation, while during the waking state, replay is thought to support both memory consolidation and retrieval processes.

Negative and positive manipulations of SWR events have demonstrated negative and positive impacts, respectively, on learning performance The disruption of SWRs during sleep after behavioral sessions impairs the subsequent learning of a memory-based task in rats (See e.g., Girardeau et al. (2009) Nat Neurosci., vol. 12, pp. 1222-1223; Ego-Stengel et al. (2010) Hippocampus, vol. 20, pp. 1-10). Electrical disruption of SWRs that occur during learning a memory-based task impairs acquisition of the task (see, e.g., Jadhav et al. (2012) Science, vol. 336, pp. 1454-8). Conversely, increased SWR activity (e.g. size, number, quantity) in rats correlates with improved performance of various memory tasks (See, e.g., Singer et al. (2013) Neuron, vol. 77, pp. 1163-73; Dupret et al. (2010) Nat Neurosci., vol. 13, pp. 995-1002). An optogenetic manipulation which lengthened hippocampal replay events caused improved learning of a memory-based task (See, e.g., Fernandez-Ruiz et al. (2019) Science, vol. 364, pp. 1082-1086). These results demonstrate that modulation of SWR events can modulate memory performance However electrical disruption and optogenetic techniques are not amenable to use in human subjects. The present disclosure describes a method for enhancing and promoting SWRs and replay that is extendable to human subjects.

Aspects of the present disclosure include a neurofeedback based training paradigm that modulates SWR activity (e.g. occurrence rate) in a subject. In some embodiments, the neurofeedback based training modulates SWR features (e.g. amplitude, duration, spectral content). In some embodiments, the methods of the present disclosure include detecting SWR activity in real-time using electrodes in the hippocampus of a subject. In some embodiments, the feedback (e.g. neurofeedback) comprises external sensory and/or reward feedback coupled to the detection of SWRs. In some embodiments, the feedback modulates hippocampal replay in the subject. In some embodiments, the feedback increases hippocampal replay in the subject.

In some embodiments, the methods of the present disclosure include recording a plurality of signals from one or more regions of the brain. In some embodiments, the plurality of signals are acquired by any known neurophysiological recording device.

In some embodiments, the plurality of signals are neural signals. In some embodiments, the neural signals are local field potentials. In some embodiments, the plurality of signals are intracranial single unit recordings. In some embodiments, the plurality of signals are recorded by a non-invasive recording device or a minimally invasive recording device. In some embodiments, the plurality of signals are recorded by a Magnetoencephalographic Imaging (MEGI) device, an Electroencephalography (EEG) device, functional magnetic resonance imaging (fMRI) device, or a Electrocorticography (ECoG) device. In some embodiments, the plurality of signals are MEGI signals, EEG signals, fMRI signals, or ECoG signals. In some embodiments, the recording device is a wearable neural detector device. In some embodiments, the recording device is an implantable recording device.

In some embodiments, the plurality of signals are recorded from one or more electrodes. In some embodiments, the plurality of signals are recorded from an electrode array. In some embodiments, the electrode array is a medial temporal lobe electrode array. The number of electrodes operably coupled to the hippocampus may be chosen so as to provide the desired resolution and information about the neurophysiological neural signals being generated in the hippocampus, for example, during one or more behavioral tasks, as each electrode may convey information about the activity of a particular region (e.g., the hippocampus, amygdala, and the prefrontal cortex thalamus (including the central thalamus), sensory cortex (including the somatosensory cortex), zona incerta, ventral tegmental area, nucleus accumbens, substantia nigra, ventral pallidum, globus pallidus, dorsal striatum, ventral striatum, subthalamic nucleus, dentate gyrus, cingulate gyrus, entorhinal cortex, olfactory cortex, primary motor cortex, cerebellum, or any combination thereof).

In some embodiments, each of the one or more electrodes include one or more “clusters” of recording electrode sites, e.g. the plurality of electrode sites on a brain tissue. Each cluster may have any particular number of electrodes. For instance, a cluster may include a stereotrode (2 closely spaced electrode sites), a tetrode (4 closely spaced electrode sites), an octrode (8 closely spaced electrode sites), or a polytrode. In some embodiments, an electrode array comprises 1 or more, 5 or more, 10 or more, 15 or more, 20 or more, 25 or more, 30 or more, 35 or more, 40 or more, 45 or more, or 50 or more tetrodes. In some embodiments, the array of electrodes is implanted into the subject. In some embodiments, the one or more electrode arrays includes approximately 10-300 separate recording electrode sites distributed among brain regions, although the electrode array may include any suitable number of recording sites. Accordingly, in some embodiments, at least 3 electrodes are employed on the brain of the subject. In some embodiments, between about 3 and 1024 electrodes, or more, may be employed. In some embodiments, the number of electrodes positioned on or in the brain of the subject is about 1 to 10 electrodes, about 10 to 20 electrodes, about 20 to 30 electrodes, about 30 to 40 electrodes, about 40 to 50 electrodes, about 60 to 70 electrodes, about 70 to 80 electrodes, about 80 to 90 electrodes, about 90 to 100 electrodes, about 100 to 110 electrodes, about 110 to 120 electrodes, about 120 to 130 electrodes, about 130 to 140 electrodes, about 140 to 150 electrodes, about 150 to 160 electrodes, about 160 to 170 electrodes, about 170 to 180 electrodes, about 180 to 190 electrodes, about 190 to 200 electrodes, about 200 to 210 electrodes, about 210 to 220 electrodes, about 220 to 230 electrodes, about 230 to 240 electrodes, about 240 to 250 electrodes, about 250 to 300 electrodes (e.g., a 16×16 array of 256 electrodes), about 300 to 400 electrodes, about 400 to 500 electrodes, about 500 to 600 electrodes, about 600 to 700 electrodes, about 700 to 800 electrodes, about 800 to 900 electrodes, about 900 to 1000 electrodes, or about 1000 to 1024 electrodes, or more. The electrodes may be homogeneous or heterogeneous.

### Positioning of Electrodes

In some embodiments, the method comprises positioning one or more electrodes on or in the brain of the subject. For example, a subject's brain may first be imaged by any convenient means, such as magnetic resonance imaging (MRI). The specific location at which to position an electrode may be determined by identification of anatomical landmarks in the subject's brain, such as the pre-central and post-central gyri and the central sulcus. Identification of anatomical landmarks in a subject's brain may be accomplished by any convenient means, such as magnetic resonance imaging (MRI), functional magnetic resonance imaging (fMRI), and visual inspection of a subject's brain while undergoing a craniotomy. Once a suitable location for an electrode is determined, the electrode may be positioned on or implanted into the brain according to any convenient means. In some embodiments, the electrodes are implanted in the medial temporal lobe. Suitable locations for positioning or implanting the electrodes may include, but are not limited to, one or more regions of hippocampus, amygdala, the prefrontal cortex, thalamus (including the central thalamus), sensory cortex (including the somatosensory cortex), zona incerta, ventral tegmental area, nucleus accumbens, substantia nigra, ventral pallidum, globus pallidus, dorsal striatum, ventral striatum, subthalamic nucleus, dentate gyrus, cingulate gyrus, entorhinal cortex, olfactory cortex, primary motor cortex, cerebellum, or any combination thereof. Correct placement of the electrodes may be confirmed by any convenient means, including visual inspection or computed tomography (CT) scan. In some aspects, after electrode positions are confirmed, they may be superimposed on a surface reconstruction image of the subject's brain. In certain aspects, the electrodes are positioned such that the plurality of signals are detected from one or more regions of the hippocampus, amygdala, the prefrontal cortex, thalamus (including the central thalamus), sensory cortex (including the somatosensory cortex), zona incerta, ventral tegmental area, nucleus accumbens, substantia nigra, ventral pallidum, globus pallidus, dorsal striatum, ventral striatum, subthalamic nucleus, dentate gyrus, cingulate gyrus, entorhinal cortex, olfactory cortex, primary motor cortex, cerebellum, or any combination thereof.

Methods of interest for positioning electrodes further include, but are not limited to, those described in U.S. Pat. Nos. 4,084,583; 5,119,816; 5,291,888; 5,361,773; 5,479,934; 5,724,984; 5,817,029; 6,256,531; 6,381,481; 6,510,340; 7,239,910; 7,715,607; 7,908,009; 8,045,775; and 8,019,142; the disclosures of which are incorporated herein by reference in their entireties for all purposes.

Electrodes may be arranged in no particular pattern or any convenient pattern to facilitate detection of neural signals. In some embodiments, an array of electrodes (e.g., an ECoG array, microelectrode array, EEG array) is positioned on the surface of the medial temporal lobe such that the array covers the entire or substantially the entire region of the hippocampus. In some embodiments, the electrodes will be placed within or through the hippocampus.

In some embodiments, the electrode is a depth electrode. In some embodiments, the depth electrode is a microwire depth electrode. Non-limiting examples of different electrode arrays and example positioning thereof can be found in U.S. Pat. Nos. 9,905,239 and 10,363,420, which are hereby incorporated by reference in their entirety.

Electrodes may be pre-arranged into an array, such that the array includes a plurality of electrodes that may be placed on or in a subject's brain. Such arrays may be miniature- or micro-arrays, a non-limiting example of which may be a miniature neurophysiological array (e.g. ECoG array, microelectrode array, EEG array). For a general review of ECoG technology, see Ajmone-Marsan, C. Electrocorticography: Historical Comments on its Development and the Evolution of its Practical Applications, Electroencephalogr. Clin. Neurophysiol, Suppl. 1998, 48: 10-16; the disclosure of which is incorporated herein by reference.

Also of interest are electrodes that may receive EEG data. One or more wet or dry

EEG electrodes may be used in practicing the subject methods. Electrodes and electrode systems of interest further include, but are not limited to, those described in U.S. Patent Publication Numbers 2007/0093706, 2009/0281408, 2010/0130844, 2010/0198042, 2011/0046502, 2011/0046503, 2011/0046504, 2011/0237923, 2011/0282231, 2011/0282232 and U.S. Pat. Nos. 4,709,702, 4967038, 5038782, 6154669; the disclosures of which are incorporated herein by reference.

In certain embodiments, the array may cover a surface area of about 1 cm², about 1 to 10 cm², about 10 to 25 cm², about 25 to 50 cm², about 50 to 75 cm², about 75 to 100 cm², or 100 cm² or more. Arrays of interest may include, but are not limited to, those described in U.S. Patent Nos. USD565735; USD603051; USD641886; and USD647208; the disclosures of which are incorporated herein by reference.

Electrodes may be platinum-iridium electrodes or be made out of any convenient material. The diameter, length, and composition of the electrodes to be employed may be determined in accordance with routine procedures known to those skilled in the art. Factors which may be weighed when selecting an appropriate electrode type may include but not be limited to the desired location for placement, the type of subject, the age of the subject, cost, duration for which the electrode may need to be positioned, and other factors.

### Behavioral Tasks

Aspects of the present methods include recording a plurality of signals from one or more electrodes during a behavioral task. In some embodiments, the method includes detecting SWR activity during behavioral tasks performed by the subject, during quiet rest, or a combination thereof. In some embodiments, said recording occurs when the subject is performing a memory-related or non-memory related behavioral task. In some embodiments, the behavioral task involves identifying thought patterns and mental strategies that increase SWR occurrence. Non-limiting examples of memory-related activities/prompts include remote episodic memory (e.g. as recounting a story from childhood), recent episodic memory (e.g. describing events earlier in the day), semantic memory (e g naming state capitols), spatial memory (e.g. describing a route of their daily commute), sequential non-spatial memory (e.g. describing steps of a familiar process), or a combination thereof. In some embodiments, the feedback occurs during non-memory-dependent activities and/or prompts. Non-limiting examples of non-memory dependent activities include, but are not limited to reading a sentence aloud, tracing a simple drawing, or doing basic arithmetic problems. In some embodiments, the one or more behavioral tasks include listening to one or more questions. In some embodiments, the one or more questions are pre-recorded questions. In some embodiments, the one or more behavioral tasks comprise reading one or more answers on a screen. In some embodiments, the one or more behavioral tasks comprise reading aloud one or more syllables, words, parts of words, phrases, utterances, paragraphs, sentences, and/or a combination thereof. In some embodiments, the one or more behavioral tasks comprise verbally producing a set of answer responses after listening to the one or more questions.

### Signal Processing

The signals received from the one or more electrodes are received and processed in real-time. Prior to the real-time processing, a signal is referred to herein as an “input signal,” regardless of whether or not the signal itself is an output from any previous step. Input signals may originate from one or more electrodes. In some embodiments, the electrodes are communicatively coupled to a processor apparatus that does the processing in real-time. The electrodes may also be in direct communication with a processor apparatus. In some embodiments, the electrodes may not be in direct physical communication with the processor, but may instead transmit the information by any convenient means. Of interest is wireless communication, a non-limiting example of which is described in US Patent Publication 2006/0129056; the disclosure of which is incorporated herein by reference.

Input signals may comprise a wide range of frequencies, which may depend upon factors including but not limited to the particular type of electrode employed, the type of subject, the position of the electrode, and other factors. In certain embodiments, an input signal may comprise frequencies of about 1 Hz to 500 Hz or more. In certain embodiments, an input signal may comprise frequencies from the range of about 1 to 10 Hz, about 10 to 20 Hz, about 20 to 30 Hz, about 30 to 40 Hz, about 40 to 50 Hz, about 50 to 60 Hz, about 60 to 70 Hz, about 70 to 80 Hz, about 80 to 90 Hz, about 90 to 100 Hz, about 100 to 125 Hz, about 125 Hz to 150 Hz, about 150 Hz to 175 Hz, about 175 Hz to 200 Hz, about 200 Hz to 225 Hz, about 225 Hz to 250 Hz, about 250 Hz to 275 Hz, about 275 Hz to 300 Hz, about 300 Hz to 325 Hz, about 325 Hz to 350 Hz, about 350 Hz to 375 Hz, about 375 Hz to 400 Hz, about 400 Hz to 425 Hz, about 425 Hz to 450 Hz, about 450 Hz to 475 Hz, or about 475 Hz to 500 Hz or more.

In some embodiments, input signals comprise delta, theta, alpha, mu, beta, gamma, or high gamma frequencies. In some embodiments, input signals comprise only one of delta, theta, alpha, mu, beta, gamma, and high gamma frequency bands. Other embodiments may comprise one or more of delta, theta, alpha, mu, beta, gamma, and high gamma frequency bands.

In some embodiments, processing the plurality of input signals of the present disclosure comprises applying one or more filters to an input signal. Aspects of the present disclosure include filtering the plurality of signals from one or more regions of the brain of the subject. In some embodiments, the plurality of signals are filtered to any frequency related to SWR (e.g. low gamma frequency range, gamma frequency range, high gamma frequency range). In some embodiments, the plurality of signals are filtered to a frequency ranging from 20-250 Hz. For example, in some embodiments, the methods of the present disclosure includes filtering the plurality of signals to a frequency ranging from 5-10 Hz, 10-20 Hz, 20-30 Hz, 30-40 Hz, 40-50 Hz, 50-60 Hz, 60-70 Hz, 70-80 Hz, 80-90 Hz, 90-1100 Hz, 100-110 Hz, 110-120 Hz, 120-130 Hz, 130-140 Hz, 140-150 Hz, 150-160 Hz, 160-170 Hz, 170-180 Hz, 180-190 Hz, 190-200 Hz, 200-210 Hz, 210-220 Hz, 220-230 Hz, 230-240 Hz, or 240-250 Hz. In some embodiments, the methods of the present disclosure includes filtering the plurality of signals to a frequency ranging from 20-50 Hz, 30-50 Hz, 50-80 Hz, 80-150 Hz, or 150-250 Hz.

In some embodiments, processing the input signal comprises filtering the plurality of signals by applying one or more notch filters, bandpass filters, a low-pass filter, or a combination thereof. In some embodiments, processing the plurality of input signals comprises approximating an envelope of a signal. Non-limiting examples of processing and/or filtering the plurality of signals is described in U.S. Patent Publication No. 2015/0313497, which is hereby incorporated by reference in its entirety.

The envelope of any input signal may be approximated prior to any other processing or filtering, or may be approximated after other filtering or processing of an input signal has already taken place. Approximating an envelope may be achieved by any convenient means, such as applying a Hilbert transform, a Fourier transform, or a low-pass filter to an absolute value of a signal. In some embodiments, approximating an envelope occurs in the absence of averaging based on time.

In some embodiments, the methods of the present disclosure include processing the plurality of signals by calculating the phase of a signal. Calculating the phase of a signal may comprise any convenient means, such as a calculation using a Hilbert transform. Processing comprising calculating the phase of a signal may include, but is not limited to, methods described by Canolty, et al. (Science, 15 Sep. 2006: Vol. 313, pp. 1626-1628), the disclosure of which is incorporated herein by reference.

In practicing methods of the invention, a subject's brain activity (e.g. SWRs in the subject) is detected, by any convenient means. In many instances, detecting a subject's brain activity comprises positioning one or more electrodes, wherein the electrode(s) are of a suitable type and position so as to detect a subject's brain activity (e.g. SWRs in the subject). In some embodiments, the method includes detecting SWR events using, for example, a percentile-based threshold rather than a standard deviation-based method. In some embodiments, SWR detection criteria may include, but is not limited to, a minimum number of cycles of oscillatory activity at a particular frequency (e.g. 3 cycles at 80-140 Hz). Other conventional techniques may be used to detect SWRs in a subject. For example, other conventional techniques can be used to detect SWRs in a subject depending on use of different recording devices (e.g. EEG device, MEGI device, fMRI device, ECoG device).

### Neurofeedback

In some embodiments, when the subject generates a SWR, the subject receives rapid sensory feedback (e.g. neurofeedback). In some embodiments, such feedback takes various forms, such as, but not limited to sharing the following general features: brief, positive, non-alarming or distracting, with unmistakable features. In some embodiments, such feedback comprises an auditory cue. In some embodiments, the auditory cue is paired with food reward. In some embodiments, such feedback comprises a visual cue. In some embodiments, such feedback comprises both an auditory and a visual cue (e.g. a gold star icon appearing on a tablet screen accompanied by a chime noise in response to SWR detection). In some embodiments, the feedback occurs in response to SWR activity during one or more behavioral tasks. In some embodiments, such feedback could occur during quiet rest.

In some embodiments, providing feedback to the subject when SWRs are detected results in increased likelihood that the subject will generate an increased amount of SWRs or achieve a SWR-conductive state as compared to the same subject prior to receiving neurofeedback training. In some embodiments, SWR occurrence following neurofeedback training results in statistically significant increase in occurrence rate as compared to occurrence rates in the patient before training began. In some embodiments, increasing the occurance of SWRs results in enhanced memory performance ability of the subject. In some embodiments, memory performance includes memory retrieval. In some embodiments, increasing the occurrence of SWRs results in enhanced memory performance ability of the subject and/or enhanced cognitive flexibility of the subject. In some embodiments, increasing the occurrence of SWRs results in enhanced memory retrieval ability of the subject and/or enhanced cognitive flexibility of the subject. Such a neurofeedback training in the methods of the present disclosure may enhance memory ability in healthy subjects and/or boost memory function from subjects suffering from cognitive decline due to aging or disease. In some embodiments, feedback increases the SWR activity in the subject. In some embodiments, increasing the occurrence of SWRs results in enhanced decision-making ability of the subject. In some embodiments, increasing the occurrence of SWRs results in improved cognitive function of the subject. Such improvements in cognitive function can be measured in a wide variety of ways using known conventional techniques.

In some embodiments, the method includes providing feedback to the subject triggered by the detection of SWR activity in the subject above a set threshold (e.g. 2 or more standard deviations (SD) above the mean envelope of the filtered neural data). In some embodiments, the set threshold is 3 or more SD above the mean envelope of the filtered neural data, 4 or more SD above the mean envelope of the filtered neural data, 5 or more SD above the mean envelope of the filtered neural data, 6 or more SD above the mean envelope of the filtered neural data, 7 or more SD above the mean envelope of the filtered neural data, 8 or more SD above the mean envelope of the filtered neural data, 9 or more SD above the mean envelope of the filtered neural data, 10 or more SD above the mean envelope of the filtered neural data, 11 or more SD above the mean envelope of the filtered neural data, 12 or more SD above the mean envelope of the filtered neural data, 13 or more SD above the mean envelope of the filtered neural data, 14 or more SD above the mean envelope of the filtered neural data, 15 or more SD above the mean envelope of the filtered neural data, 16 or more SD above the mean envelope of the filtered neural data, 17 or more SD above the mean envelope of the filtered neural data, 18 or more SD above the mean envelope of the filtered neural data, 19 or more SD above the mean envelope of the filtered neural data, or 20 or more SD above the mean envelope of the filtered neural data. In some embodiments, the methods and systems of the present disclosure comprise gradually increasing the set threshold for SWR detection over the course of training (e.g from 2SD to 20SD above mean).

In some embodiments, the methods of the present disclosure are carried out using a receiver unit, comprising: a receiver in communication with a transmitter that receives the plurality of signals detected from the at least three electrodes; one or more processors; a non-transient computer-readable medium comprising instructions that, when executed by the one or more processors, cause the one or more processors to: perform one or more filters on the plurality of signals; and detect SWR activity using one or more methods of the present disclosure. In some embodiments, the method further comprises instructions that, when executed by the one or more processors, cause the one or more processors to send sensory feedback to the subject. In some embodiments, the feedback is sent via tablet, computer, or phone.

### Systems

Aspects of the present disclosure include systems to carry out the methods as described herein.

Aspects of the present disclosure include systems for enhancing and/or treating memory performance in a subject, the system comprising: an electrode array in contact with one or more regions of the brain of the subject; an electrical recording device configured to record a plurality of signals in the one or more regions of the brain; one or more processors, a non-transient computer-readable medium comprising instructions that, when executed by the processor, cause the processor to: perform one or more filters on the plurality of signals to filter the plurality of signals to a frequency ranging from 20-250 Hz; detect sharp wave ripple (SWR) activity in the subject from the filtered signals that exceed a set threshold; a feedback source configured to provide feedback to the subject triggered by the detection of SWR activity, wherein the feedback to the subject is configured to increase SWR activity in the subject as compared to the same subject prior to training, wherein the increase in SWR activity results in enhanced or increased memory performance.

Aspects of the present disclosure further include systems that modulate hippocampal replay and/or SWR activity in a subject, the system comprising: an electrode array in contact with one or more regions of the brain of the subject; an electrical recording device configured to record a plurality of signals in the one or more regions of the brain; one or more processors, a non-transient computer-readable medium comprising instructions that, when executed by the processor, cause the processor to: perform one or more filters on the plurality of signals to filter the plurality of signals to a frequency ranging from 20-250 Hz; detect sharp wave ripple (SWR) activity in the subject from the filtered signals that exceed a set threshold; a feedback source configured to provide feedback to the subject triggered by the detection of SWR activity, wherein the feedback to the subject is configured to modulate SWR activity in the subject.

In some embodiments, the region of the brain in which brain activity (e.g. SWR activity) is recorded is in the hippocampus, amygdala, the prefrontal cortex, thalamus (including the central thalamus), sensory cortex (including the somatosensory cortex), zona incerta, ventral tegmental area, nucleus accumbens, substantia nigra, ventral pallidum, globus pallidus, dorsal striatum, ventral striatum, subthalamic nucleus, dentate gyrus, cingulate gyrus, entorhinal cortex, olfactory cortex, primary motor cortex, cerebellum, or any combination thereof. In some embodiments, the region of the brain is the hippocampus. In some embodiments, the region of the brain is a brain region associated with memory.

The systems and methods of the present disclosure include one or more electrodes. In some embodiments, recording a plurality of signals of the present methods is recorded from an electrode array. In some embodiments, the electrode array is a medial temporal lobe electrode array. The number of electrodes operably coupled to the hippocampus may be chosen so as to provide the desired resolution and information about the neurophysiological neural signals being generated in the hippocampus, for example, during one or more behavioral tasks, as each electrode may convey information about the activity of a particular region (e.g., the hippocampus, amygdala, and the prefrontal cortex).

In some embodiments, each of the one or more electrodes include one or more “clusters” of recording electrode sites, although the plurality of electrode sites on a brain tissue. Each cluster may have any particular number of electrodes. For instance, a cluster may include a stereotrode (2 closely spaced electrode sites), a tetrode (4 closely spaced electrode sites), an octrode (8 closely spaced electrode sites), or a polytrode. In some embodiments, an electrode array comprises 1 or more, 5 or more, 10 or more, 15 or more, 20 or more, 25 or more, 30 or more, 35 or more, 40 or more, 45 or more, or 50 or more tetrodes). In some embodiments, the array of electrodes is implanted into the subject. In some embodiments, the array of electrodes is implanted in the medial temporal lobe. In some embodiments, the one or more electrode arrays includes approximately 10-300 separate recording electrode sites distributed among brain regions, although the electrode array may include any suitable number of recording sites. Accordingly, in some embodiments, the electrodes are employed on or in a region of the brain. In some embodiments, between about 3 and 1024 electrodes, or more, may be employed. In some embodiments, the number of electrodes positioned is about 1 to 10 electrodes, about 10 to 20 electrodes, about 20 to 30 electrodes, about 30 to 40 electrodes, about 40 to 50 electrodes, about 60 to 70 electrodes, about 70 to 80 electrodes, about 80 to 90 electrodes, about 90 to 100 electrodes, about 100 to 110 electrodes, about 110 to 120 electrodes, about 120 to 130 electrodes, about 130 to 140 electrodes, about 140 to 150 electrodes, about 150 to 160 electrodes, about 160 to 170 electrodes, about 170 to 180 electrodes, about 180 to 190 electrodes, about 190 to 200 electrodes, about 200 to 210 electrodes, about 210 to 220 electrodes, about 220 to 230 electrodes, about 230 to 240 electrodes, about 240 to 250 electrodes, about 250 to 300 electrodes (e.g., a 16×16 array of 256 electrodes), about 300 to 400 electrodes, about 400 to 500 electrodes, about 500 to 600 electrodes, about 600 to 700 electrodes, about 700 to 800 electrodes, about 800 to 900 electrodes, about 900 to 1000 electrodes, or about 1000 to 1024 electrodes, or more. The electrodes may be homogeneous or heterogeneous.

The specific location at which to position an electrode may be determined by identification of anatomical landmarks in the subject's brain, such as the pre-central and post-central gyri and the central sulcus. Identification of anatomical landmarks in a subject's brain may be accomplished by any convenient means, such as magnetic resonance imaging (MRI), functional magnetic resonance imaging (fMRI), and visual inspection of a subject's brain while undergoing a craniotomy. Once a suitable location for an electrode is determined, the electrode may be positioned (e.g., implanted) according to any convenient means. Suitable locations for positioning or implanting the electrodes may include, but are not limited to, one or more regions of hippocampus, amygdala, the prefrontal cortex, thalamus (including the central thalamus), sensory cortex (including the somatosensory cortex), zona incerta, ventral tegmental area, nucleus accumbens, substantia nigra, ventral pallidum, globus pallidus, dorsal striatum, ventral striatum, subthalamic nucleus, dentate gyms, cingulate gyrus, entorhinal cortex, olfactory cortex, primary motor cortex, cerebellum, or any combination thereof. . Correct placement of the at least three electrodes may be confirmed by any convenient means, including visual inspection or computed tomography (CT) scan. In some aspects, after electrode positions are confirmed, they may be superimposed on a surface reconstruction image of the subject's brain. In certain aspects, the electrodes are positioned such that the neurophysiological signals are detected from one or more regions of the hippocampus, amygdala, the prefrontal cortex, thalamus (including the central thalamus), sensory cortex (including the somatosensory cortex), zona incerta, ventral tegmental area, nucleus accumbens, substantia nigra, ventral pallidum, globus pallidus, dorsal striatum, ventral striatum, subthalamic nucleus, dentate gyrus, cingulate gyms, entorhinal cortex, olfactory cortex, primary motor cortex, cerebellum, or any combination thereof.

Methods of interest for positioning electrodes further include, but are not limited to, those described in U.S. Pat. Nos. 4,084,583; 5,119,816; 5,291,888; 5,361,773; 5,479,934; 5,724,984; 5,817,029; 6,256,531; 6,381,481; 6,510,340; 7,239,910; 7,715,607; 7,908,009; 8,045,775; and 8,019,142; the disclosures of which are incorporated herein by reference in their entireties for all purposes.

Electrodes may be arranged in no particular pattern or any convenient pattern to facilitate detection of neural signals. In some embodiments, an array of electrodes (e.g., an ECoG array, microelectrode array, EEG array) is positioned on the surface of the hippocampus such that the array covers the entire or substantially the entire region of the hippocampus. In some embodiments, the electrodes will be placed within or through the hippocampus.

In some embodiments, the electrode is a depth electrode. In some embodiments, the depth electrode is a microwire depth electrode. Non-limiting examples of an array and example positioning thereof can be found in U.S. Pat. Nos. 9,905,239 and 10,363,420, which are hereby incorporated by reference in their entirety.

Electrodes may be pre-arranged into an array, such that the array includes a plurality of electrodes that may be placed on or in a subject's brain. Such arrays may be miniature- or micro-arrays, a non-limiting example of which may be a miniature neurophysiological array (e.g. ECoG array, microelectrode array, EEG array). For a general review of ECoG technology, see Ajmone-Marsan, C. Electrocorticography: Historical Comments on its Development and the Evolution of its Practical Applications, Electroencephalogr. Clin. Neurophysiol, Suppl. 1998, 48: 10-16; the disclosure of which is incorporated herein by reference.

Also of interest are electrodes that may receive EEG data. One or more wet or dry EEG electrodes may be used in practicing the subject methods. Electrodes and electrode systems of interest further include, but are not limited to, those described in U.S. Patent Publication Numbers 2007/0093706, 2009/0281408, 2010/0130844, 2010/0198042, 2011/0046502, 2011/0046503, 2011/0046504, 2011/0237923, 2011/0282231, 2011/0282232 and U.S. Pat. Nos. 4,709,702, 4967038, 5038782, 6154669; the disclosures of which are incorporated herein by reference.

In certain embodiments, the array may cover a surface area of about 1 cm², about 1 to 10 cm², about 10 to 25 cm², about 25 to 50 cm², about 50 to 75 cm², about 75 to 100 cm², or 100 cm² or more. Arrays of interest may include, but are not limited to, those described in U.S. Patent Nos. USD565735; USD603051; USD641886; and USD647208; the disclosures of which are incorporated herein by reference.

Electrodes may be platinum-iridium electrodes or be made out of any convenient material. The diameter, length, and composition of the electrodes to be employed may be determined in accordance with routine procedures known to those skilled in the art. Factors which may be weighed when selecting an appropriate electrode type may include but not be limited to the desired location for placement, the type of subject, the age of the subject, cost, duration for which the electrode may need to be positioned, and other factors.

In some embodiments, the plurality of signals of the present disclosure are neural signals. In some embodiments, the plurality of signals are local field potentials. In some embodiments, the plurality of signals are intracranial single unit recordings. In some embodiments, the plurality of signals are recorded by a non-invasive recording device or a minimally invasive recording device. In some embodiments, the plurality of signals are recorded by a Magnetoencephalographic Imaging (MEGI) device, an Electroencephalography (EEG) device, a functional magnetic resonance imaging (fMRI) device, or a Electrocorticography (ECoG) device. In some embodiments, the plurality of signals are MEGI signals, EEG signals, fMRI signals, or ECoG signals.

### Signal Processing

The signals received from the one or more electrodes are received and processed in real-time. In certain embodiments, the electrodes are communicatively coupled to one or more processors that does the processing in real-time. The electrodes may also be in direct communication with one or more processors. In certain aspects, the electrodes may not be in direct physical communication with the processor, but may instead transmit the information by any convenient means. Of interest is wireless communication, a non-limiting example of which is described in US Patent Publication 2006/0129056; the disclosure of which is incorporated herein by reference.

Input signals may comprise a wide range of frequencies, which may depend upon factors including but not limited to the particular type of electrode employed, the type of subject, the position of the electrode, and other factors. In certain embodiments, an input signal may comprise frequencies of about 1 Hz to 500 Hz or more. In certain embodiments, an input signal may comprise frequencies from the range of about 1 to 10 Hz, about 10 to 20 Hz, about 20 to 30 Hz, about 30 to 40 Hz, about 40 to 50 Hz, about 50 to 60 Hz, about 60 to 70 Hz, about 70 to 80 Hz, about 80 to 90 Hz, about 90 to 100 Hz, about 100 to 125 Hz, about 125 Hz to 150 Hz, about 150 Hz to 175 Hz, about 175 Hz to 200 Hz, about 200 Hz to 225 Hz, about 225 Hz to 250 Hz, about 250 Hz to 275 Hz, about 275 Hz to 300 Hz, about 300 Hz to 325 Hz, about 325 Hz to 350 Hz, about 350 Hz to 375 Hz, about 375 Hz to 400 Hz, about 400 Hz to 425 Hz, about 425 Hz to 450 Hz, about 450 Hz to 475 Hz, or about 475 Hz to 500 Hz or more.

In some embodiments, input signals comprise gamma or high gamma frequencies. Certain embodiments may comprise only one of gamma and high gamma frequency bands. Other embodiments may comprise one or more of gamma and high gamma frequency bands.

Aspects of the present disclosure include a non-transient computer-readable medium comprising instructions that, when executed by the one or more processors, cause the processor to process the plurality of signals in order to detect SWR activity in the subject.

In some embodiments, the systems of the present disclosure comprise a receiver unit. In some embodiments, the receiver unit is in communication with a wireless transmitter that receives the plurality of signals. In some embodiments, the receiver unit comprises one or more processors. In some embodiments, the receiver unit comprises a non-transient computer-readable medium comprising instructions which, when executed by one or more processors, causes the processor to process the plurality of signals recorded on the recording device.

Aspects of the present disclosure include a a non-transient computer-readable medium comprising instructions which, when executed by a processor, carry out the signal processing of the plurality of signals recorded on the recording device, as described herein. In some embodiments, the non-transient computer-readable medium comprising instructions which, when executed by the one or more processors, causes the processor to apply one or more filters on the plurality of signals.

In some embodiments, the plurality of input signals of the present disclosure are processed by applying one or more filters to an input signal. Aspects of the present disclosure include filtering the plurality of signals from one or more regions of the brain of the subject. In some embodiments, the plurality of signals are filtered to any frequency related to SWR (e.g. low gamma frequency range, gamma frequency range, high gamma frequency range). In some embodiments, the plurality of signals are filtered to a frequency ranging from 5-10 Hz, 10-20 Hz, 20-250 Hz. For example, in some embodiments, the plurality of signals are filtered to a frequency ranging from 30-40 Hz, 40-50 Hz, 50-60 Hz, 60-70 Hz, 70-80 Hz, 80-90 Hz, 90-100 Hz, 100-110 Hz, 110-120 Hz, 120-130 Hz, 130-140 Hz, 140-150 Hz, 150-160 Hz, 160-170 Hz, 170-180 Hz, 180-190 Hz, 190-200 Hz, 200-210 Hz, 210-220 Hz, 220-230 Hz, 230-240 Hz, or 240-250 Hz. In some embodiments, the plurality of signals are filtered to a frequency ranging from 20-50 Hz, 30-50 Hz, 50-80 Hz, 80-150 Hz, or 150-250 Hz.

In some embodiments, the input signal is processed by applying a filter on the plurality of signals, such as, but not limited to one or more notch filters, bandpass filters, a low-pass filter, or a combination thereof. In some embodiments, the plurality of input signals are processed by approximating an envelope of a signal. Non-limiting examples of processing and/or filtering the plurality of signals is described in U.S. Patent Publication No. 2015/0313497, which is hereby incorporated by reference in its entirety.

The envelope of any input signal may be approximated prior to any other processing or filtering, or may be approximated after other filtering or processing of an input signal has already taken place. Approximating an envelope may be achieved by any convenient means, such as applying a Hilbert transform, a Fourier transform, or a low-pass filter to an absolute value of a signal. In many embodiments, approximating an envelope occurs in the absence of averaging based on time.

In some embodiments, the computer-readable medium (e.g. non-transient computer readable medium) comprises instructions that, when executed by the processor, cause the processor to calculate the phase of a signal. Calculating the phase of a signal may comprise any convenient means, such as a calculation using a Hilbert transform. In some embodiments, calculating the phase of a signal may include, but is not limited to, methods described by Canolty, et al. (Science, 15 Sep. 2006: Vol. 313, pp. 1626-1628), the disclosure of which is incorporated herein by reference.

In some embodiments, the computer-readable medium comprises instructions that, when executed by the processor, cause the processor to detect SWR activity in the subject from the filtered signals. In some embodiments, the SWR activity in the subject is detected if the filtered signals exceed a set threshold. In some embodiments, the one or more processors detect SWR events using, for example, a percentile-based threshold rather than a standard deviation-based method. In some embodiments, SWR detection criteria may include, but is not limited to, a minimum number of cycles of oscillatory activity at a particular frequency (e.g. 3 cycles at 80-140 Hz). Other conventional techniques may be used to detect SWRs in a subject. For example, different conventional techniques can be used to detect SWRs in a subject when using different recording devices (e.g. EEG device, MEGI device, fMRI device, ECoG device).

### Behavioral Tasks

In some embodiments, the feedback occurs during one or more behavioral tasks as described herein. In some embodiments, the one or more behavioral tasks is a non-memory related task or a memory-related task, as described in the methods of the present disclosure. In some embodiments, a behavioral task comprises identifying thought patterns and mental strategies that increase SWR occurrence.

In some embodiments, the plurality of signals are recorded when the subject is performing a behavioral task, during quiet rest, or a combination thereof. In some embodiments, the plurality of signals are recorded from the recording device when the subject is quietly resting. In some embodiments, the plurality of signals are recorded when the subject is performing a behavioral task.

In some embodiments, SWR activity is detected during behavioral tasks performed by the subject, during quiet rest, or a combination thereof. In some embodiments, said recording occurs when the subject is performing a memory-related or non-memory related behavioral task. In some embodiments, the behavioral task involves identifying thought patterns and mental strategies that increase SWR occurrence. Non-limiting examples of memory-related activities/prompts include remote episodic memory (e.g. as recounting a story from childhood), recent episodic memory (e.g. describing events earlier in the day), semanic memory (e g naming state capitols), spatial memory (e.g. describing a route of their daily commute), sequential non-spatial memory (e.g. describing steps of a familiar process), or a combination thereof. In some embodiments, the feedback occurs during non-memory-dependent activities and/or prompts. Non-limiting examples of non-memory dependent activities include, but are not limited to reading a sentence aloud, tracing a simple drawing, or doing basic arithmetic problems. In some embodiments, the one or more behavioral tasks include listening to one or more questions. In some embodiments, the one or more questions are pre-recorded questions. In some embodiments, the one or more behavioral tasks comprise reading one or more answers on a screen. In some embodiments, the one or more behavioral tasks comprise reading aloud one or more syllables, words, parts of words, phrases, utterances, paragraphs, sentences, and/or a combination thereof. In some embodiments, the one or more behavioral tasks comprise verbally producing a set of answer responses after listening to the one or more questions.

### Neurofeedback

Aspects of the present disclosure include a feedback source configured to provide feedback (e.g. sensory neurofeedback) to the subject triggered by the detection of SWR activity.

In some embodiments, the feedback source comprises a sensory feedback from the feedback source. In some embodiments, the feedback source comprises one or more processors. In some embodiments, the feedback is sent from the feedback source comprising a tablet, computer, or a phone. In some embodiments, the feedback comprises external sensory and/or reward feedback coupled to the detection of SWRs. In some embodiments, a computer-readable medium comprises instructions that, when executed by the one or more processors, cause the processor to provide feedback to the subject triggered by the detection of SWR activity. In some embodiments, a computer-readable medium comprises instructions that, when executed by the one or more processors, cause the processor to provide feedback to the subject triggered by the detection of SWR activity during a behavioral task.

In some embodiments, the feedback to the subject is configured to increase SWR activity in the subject relative to the SWR activity in the same subject prior to receiving said feedback. In some embodiments, the increase in SWR activity results in enhanced or increased memory performance. In some embodiments, the increase in SWR activity results in enhanced or increased memory retrieval. For example, in some embodiments, the electrical recording device of the present disclosure will record a plurality of signals in one or more regions of the brain of the subject, and the processor will process the plurality of signals as described herein to detect SWR activity in the subject without a feedback source configured to provide feedback to the subject, as a control. Following recording and processing the plurality of signals without providing feedback to the subject, recording and processing is carried out again, but now providing feedback to the subject triggered by the detection of SWR activity. In some embodiments, the feedback to the subject is configured to increase SWR activity in the subject as compared to the same subject without said feedback. In some embodiments, the feedback to the subject is configured to increase SWR activity in the subject as compared to a different subject without said feedback.

In some embodiments, the feedback from the feedback source is configured to modulate SWR activity in the subject. In some embodiments, the feedback from the feedback source is configured to modulate hippocampal replay in the subject. In some embodiments, feedback from the feedback source is configured to increase SWR occurrence in the subject. In some embodiments, feedback from the feedback source is configured to increase SWR activity in the subject. In some embodiments, feedback from the feedback source is configured to increase the size of SWRs in the subject.

Aspects of the present disclosure include a feedback source configured to provide feedback to the subject triggered by the detection of SWR activity in the subject above a set threshold (e.g. 2 or more SD above the mean envelope of the filtered neural data). In some embodiments, the set threshold is 3 or more SD above the mean envelope of the filtered neural data, 4 or more SD above the mean envelope of the filtered neural data, 5 or more SD above the mean envelope of the filtered neural data, 6 or more SD above the mean envelope of the filtered neural data, 7 or more SD above the mean envelope of the filtered neural data, 8 or more SD above the mean envelope of the filtered neural data, 9 or more SD above the mean envelope of the filtered neural data, 10 or more SD above the mean envelope of the filtered neural data, 11 or more SD above the mean envelope of the filtered neural data, 12 or more SD above the mean envelope of the filtered neural data, 13 or more SD above the mean envelope of the filtered neural data, 14 or more SD above the mean envelope of the filtered neural data, 15 or more SD above the mean envelope of the filtered neural data, 16 or more SD above the mean envelope of the filtered neural data, 17 or more SD above the mean envelope of the filtered neural data, 18 or more SD above the mean envelope of the filtered neural data, 19 or more SD above the mean envelope of the filtered neural data, or 20 or more SD above the mean envelope of the filtered neural data. In some embodiments, the methods and systems of the present disclosure comprise gradually increasing the set threshold for SWR detection over the course of training (e.g from 2 SD to 20 SD above mean envelope of the filtered neural data).

Aspects of the present disclosure include a non-transitory computer readable medium storing instructions that, when executed by a computing device (e.g. a processor), cause the computing device to perform the steps for modulating memory performance, memory retrieval, and/or hippocampal replay in a subject, as provided herein.

By “processor”, as used herein, is meant any hardware and/or software combination that will perform he functions required of it. For example, any data processor herein may be a programmable digital microprocessor such as available in the form of an electronic controller, mainframe, server or personal computer (desktop or portable). Where the data processor is programmable, suitable programming can be communicated from a remote location to the data processor, or previously saved in a computer program product (such as a portable or fixed computer readable storage medium, whether magnetic, optical or solid-state device based).

Substantially any circuitry can be configured to a functional arrangement within the systems for performing the methods disclosed herein. The hardware architecture of such circuitry, including e.g., a specifically configured computer, is well known by a person skilled in the art, and can comprise hardware components including one or more processors (CPU), a random-access memory (RAM), a read-only memory (ROM), an internal or external data storage medium (e.g., hard disk drive). Such circuitry can also comprise one or more graphic boards for processing and outputting graphical information to display means. The above components can be suitably interconnected via a bus within the circuitry, e.g., inside a specific-use computer. The circuitry can further comprise suitable interfaces for communicating with general-purpose external components such as a monitor, keyboard, mouse, network, etc. In some embodiments, the circuitry can be capable of parallel processing or can be part of a network configured for parallel or distributive computing to increase the processing power for the present methods and programs. In some embodiments, the program code read out from the storage medium can be written into a memory provided in an expanded board inserted in the circuitry, or an expanded unit connected to the circuitry, and a CPU or the like provided in the expanded board or expanded unit can actually perform a part or all of the operations according to the instructions of the programming, so as to accomplish the functions described.

### Utility

The subject methods and systems find use in any application in which it is desirable to increase and/or enhance memory performance and/or retrieval and/or cognitive flexibility in the subject. Subjects of interest include those suffering from memory loss associated with normal aging and disease. Non-limiting examples of such subjects include, but are not limited to, subjects who may be suffering from Alzheimer's disease, dementia, epilepsy, and seizures.

A SWR-based neural feedback training system of the present disclosure can be applied in the context of patients receiving medial temporal lobe grid and depth electrode arrays for the purpose of seizure monitoring for medically refractory epilepsy. During their extended stay in hospital for monitoring (approximately a week), subjects could receive sensory feedback triggered by the detection of SWRs during a range of behavioral tasks and quiet rest. The patient would be informed that patterns of neural activity related to memory processes were being detected and triggering the feedback, and that the patient should attend to and note mental state when such events occur.

Such feedback could take various forms, all sharing the following general features:

brief, positive, non-alarming or distracting, and unmistakable. For instance, SWR detection could trigger a gold star icon appearing on a tablet screen accompanied by a chime noise. Such feedback could occur during quiet rest while the patient was instructed to simply let the mind wander and see if he/she could identify thought patterns or mental strategies that increased SWR occurrence. Alternatively, the patient could participate in a more structured task which would involve, in addition to short periods of quiet rest, several minute long blocks of diverse memory- related activities. For instance, these might include activities/prompts such as recounting a story from childhood (remote episodic memory), describing events earlier in the day (recent episodic memory), naming state capitols (semanic memory), describing a route of their daily commute (spatial memory), and describing steps of a familiar process (sequential non-spatial memory). Non-memory-dependent prompts would also be interspersed, such as reading a sentence aloud, tracing a simple drawing, or doing basic arithmetic problems. SWR-triggered feedback would be provided throughout. The memory-engaging prompts would be expected to cause increase SWR occurrence, while less would be expected during non-memory-engaged tasks. The structured progression through prompts with varying memory engagement could promote exploration and engagement or various mental states, allowing the patient to experience and notice trends in mental state conducive to SWRs. With training, the subject could become more adept at engaging a SWR-conducive state and generating more SWRs.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g. amounts, rates, etc.) but some experimental errors and deviations should be accounted for.

### Example 1

### Operant Conditioning of Hippocampal Sharp Wave Ripples

Nearly every decision is influenced and informed by prior experience. This would not be possible without the hippocampus, which is critical for rapidly encoding rich, multimodal representations of experience and coordinating the long-term storage and later retrieval of these experiences. A candidate mechanism thought to contribute to both consolidation and retrieval processes is hippocampal replay, during which the neural ensemble corresponding to an experience is reactivated in a time-compressed manner Replay events typically coincide with distinctive, 150-250 Hz oscillations in the hippocampal local field potential known as sharp wave ripples (SWRs), which can be detected as a proxy for replay. Replay during sleep generally represents recent past experience and is thought to facilitate memory consolidation. However, replay in the awake state can be predictive of upcoming movement trajectory or correct choice, and awake SWR disruption acutely impairs acquisition and performance of a spatial memory task. These findings suggest that awake replay may not simply promote consolidation, but could also underlie planning or deliberation—potentially allowing the recall of previous experience to shape upcoming decisions.

To dissect the contribution of awake SWRs to memory-guided behavior, an operant conditioning task was developed that requires a rat to generate SWRs preceding the choice point in a spatial memory task. Bilateral hippocampal CA1 tetrodes were used to detect SWRs in real time and rapid auditory and reward feedback was provided when SWR detection criteria was met. Over the course of training, it was found that animals learned to reliably generate SWRs at the required stage of each trial. As animals were challenged to generate higher amplitude SWRs across training days, they responded by increasing SWR prevalence approximately two-fold across events of all amplitudes. Many of these SWRs contained clear, task-relevant replay trajectories, suggesting that they could contribute to or reflect ongoing behavior. This result demonstrates the ability to promote physiologically relevant hippocampal SWRs using real-time neurofeedback.

### Example 2

### SWRs in Memory-Based Learning

As a primary symptom of normal aging and diseases of aging such as Alzheimer's disease, memory loss is a major cause of reduced quality of life and decreased independence for millions of adults worldwide. However, effective therapies are lacking to treat or prevent memory loss. Memory is known to critically depend on the hippocampus, a highly conserved subcortical brain structure found in all mammalian species. Decades of research have linked the hippocampus in humans and in model organisms to diverse aspects of memory, including but not limited to the initial encoding of experience, the storage and consolidation of the neural representation of experience, and the retrieval of past experience to guide future actions. Although many of the neural correlates of memory are unknown, one pattern of hippocampal activity has been reliably linked with several stages of memory processing. This phenomenon, termed hippocampal replay, refers to brief events when the neural ensembles corresponding to prior experiences are reactivated in a time-compressed manner, recapitulating the neural representation of the original experiences (Wilson et al. (1994) Science, vol. 265, pp. 676-679). Hippocampal replay has been observed and linked to memory processes in diverse species including mice, rats, bats, primates, and humans. Replay tends to occur during sharp wave ripples (SWRs), distinctive high frequency fluctuations in the hippocampal local field potential. The detection of SWRs using hippocampal electrodes has become a common proxy for detecting replay events (Buzsaki et al., (2015) Hippocampus 25(10): 1073-1188)

Many pieces of evidence indicate that replay plays an important role in memory processes. Replay and SWRs are commonly observed during slow wave sleep, and during this state, they are thought to promote memory consolidation (Lee et al. (2002) Neuron, vol. 36, pp. 1183-1194). The disruption of SWRs during sleep impairs the learning of a memory-based task in rats (Girardeau et al. (2009) Nat Neurosci., vol. 12, pp. 1222-1223; Ego-Stengel et al. (2010) Hippocampus, vol. 20, pp. 1-10). In the awake state, replay and SWRs occur during pauses in ongoing behaviors (Karlsson et al. Nat Neurosci, (2009) vol. 12, pp. 913-8; O'Neill et al. (2006) Neuron, vol. 49, pp. 143-155). At these times, they may support memory consolidation but are thought to additionally contribute to memory retrieval (Joo et al. (2018) Nat Rev Neurosci, vol. 19, pp. 744-757). During behavior, studies in rats have demonstrated a correlation between increased quantity or intensity of replay events and better memory performance (Singer et al. (2013) Neuron, vol. 77, pp. 1163-73; Dupret et al. (2010) Nat Neurosci., vol. 13, pp. 995-1002). Electrical disruption of SWRs during a memory-based task impairs learning of the task (Jadhav et al. (2012) Science, vol. 336, pp. 1454-8; Nokia et al. (2012) Front Behav Neurosci, vol. 6, p. 84), demonstrating the necessity for SWRs in memory-based learning. Conversely, an experimental manipulation that lengthened replay events showed improved learning of a memory-based task (Fernandez-Ruiz et al. (2019) Science, vol. 364, pp. 1082-1086). Together, these manipulation studies show that replay is necessary for learning and that enhancing replay is sufficient to increase learning of a memory task.

Studies of aging and disease in rodent models have provide complimentary evidence linking replay with memory ability. SWR deficits have been noted in several diverse rodent models of Alzheimer's disease concurrent with memory-specific cognitive decline (Gillespie et al. (2016) Neuron, vol. 90, pp. 740-51; Iaccarino et al. (2016) Nature, vol. 540, pp. 230-235). Abnormalities in the fidelity of replay (the consistency with which it represents the original experience) have been observed in both aged animals and in a mouse model of tauopathy (Gerrard et al. (2008) J Neurosci, vol. 28, pp. 7883-90). These findings support the hypothesis that the breakdown of replay contributes to cognitive decline during aging and dementia.

While limited in number, human studies have largely corroborated the rodent findings and suggest that the role of replay in memory may be well conserved from rodents to humans. Since recording technologies in humans are largely limited to large site area electrodes that are suboptimal for the detection and isolation of single neuron signals, the analysis of replay has focused predominantly on SWRs. In humans, SWRs can be detected during non-REM sleep stages and are coordinated with cortical oscillations such as spindles and up-states, supporting the idea of privileged information transfer between the hippocampus and cortex during SWR times (Staresina et al. (2015) Nat Neurosci, vol. 18, pp. 1679-1686). Further, the number of SWRs during a post-learning nap predicted how well subjects remembered previously presented information, directly linking these events with memory function (Axmacher et al. (2008) Brain, vol. 131, pp. 1806-17). Similarly, work in humans has shown that SWR rate increases and coordination between hippocampal and cortical SWRs increases during correct trials in a memory-based task (Vaz et al. (2019) Science, vol. 363, pp. 975-978). In addition to the links between human SWRs and memory abilities, a recent technique that applied machine learning to magnetoencephalography (MEG) data was able to detect sequential replay content in human hippocampus as subjects learned a memory task (Liu et al. (2019) Cell 178(3): pp. 640-652). Together, these findings suggest a highly conserved role for replay and SWRs in memory consolidation and retrieval processes in humans.

Overall, the relationship between replay and memory performance, the impairment of replay during aging and diseases of aging concurrent with cognitive decline, and the engagement of SWRs in human memory processes together strongly support the hypothesis that SWRs play a critical role in supporting memory processes. Therefore, the prospect of improving or promoting SWRs to combat memory loss or to enhance memory ability is a compelling and novel therapeutic strategy. However, SWRs are physiologically complex, sporadic patterns of network activity that engage diverse populations of neurons—thus challenging to manipulate while preserving their native function. Current methods of rapidly manipulating cellular populations are neither sufficiently flexible nor nuanced, and cannot currently be implemented in humans. In order to address this challenge and modulate SWRs in a physiologically relevant manner extensible to humans, an operant conditioning paradigm has been designed that provides external sensory and reward feedback coupled to the detection of SWRs in order to increase their occurrence rate.

In order to assess the viability of this strategy, proof-of-concept studies were performed in rats. The approach demonstrates that animals can use external feedback triggered by SWR generation in order to adaptively modulate their own patterns of neural activity (SWRs). When challenged to produce SWRs of increasingly large magnitude, the rats learn to increase the occurrence rate of SWRs, approximately doubling their baseline rate across all magnitudes of events. Importantly, the SWRs generated during trial phases when the animal is required to produce them are indistinguishable from SWRs produced at other trial phases, including with respect to their content (representation of prior experience). Such an increase in SWR rate may represent enhanced memory retrieval and has the potential to influence memory-based behavior.

### Experimental Details

Rats perform this behavior in an enclosed maze environment. The maze is equipped with 11 ports, each of which releases milk reward when the infrared sensor detects the rat's nose and when the ports are visited in the correct order. First, rats are pre-trained on a simplified version of the spatial memory task. Each trial of the simplified task include port visits in the following order: first, to the home port, then to one of the central ports (whichever one illuminates), then to an outer arm port, then back to home to begin the next trial. At the central ports, rats must maintain a still position with nose in the port for a delay period, which begins at the initial nose entry and ends after a randomized amount of time and is indicated by an auditory cue and delivery of milk reward. Only one out of the eight outer arm ports will provide reward, such that the animal samples different arms on early trials until discovering the goal arm, then subsequently remembers and returns to that arm for optimal reward receipt. After an unpredictable number of visits, the goal arm changes, and the rat must find and remember a new goal location.

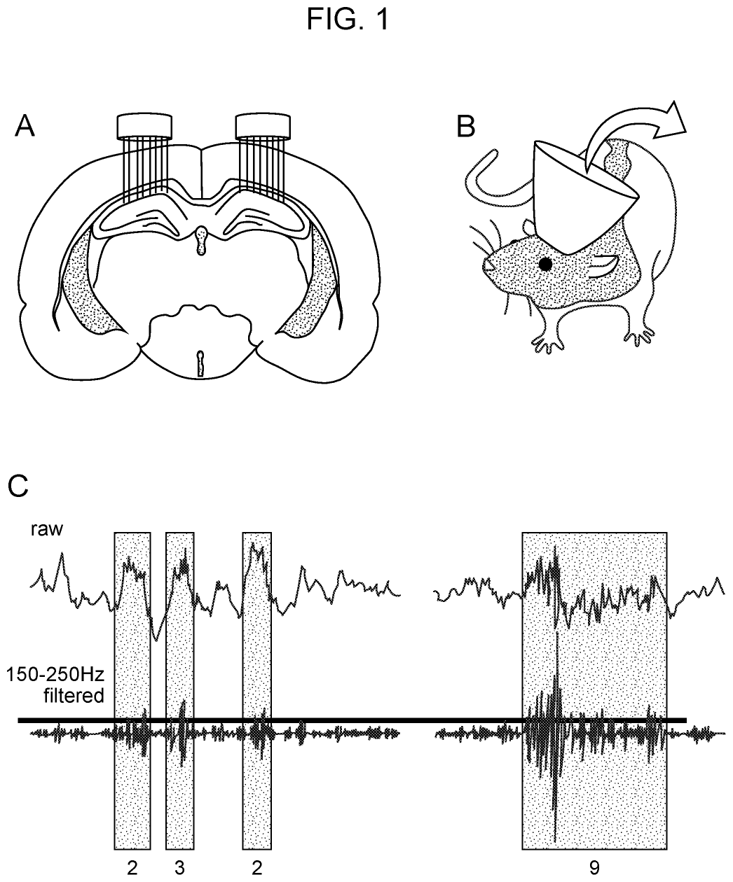

After learning the simplified task, rats are implanted with 30 independently movable bundles of 4 electrodes (tetrodes). During the recovery period post implantation, tetrodes are gradually lowered until they reach the dorsal hippocampal CA1 cell layer and detect SWRs and single neurons. At this time, rats begin the conditioning phase of the behavioral task. Data from 4-6 tetrodes are filtered for ripple-band (150-250 Hz) signal, and SWRs are detected as times when the envelope of the filtered trace exceeds a set threshold (e.g. 4 standard deviations (SD) above baseline) simultaneously on multiple tetrodes. During the conditioning phase, one of the two central ports serves as the SWR port and the other as the Control port. On trials when the SWR port illuminates (SWR trials), the rat is required to remain there until it generates a sufficiently large SWR in order to receive an auditory cue and reward (FIG. 1B). On Control trials, the rat must remain for a certain amount of time at the Control port, irrespective of SWR event detection, to receive reward; delay times are drawn from a pool of recent SWR trials to ensure that time spent at the two ports is matched. Over subsequent training days, the SWR threshold is gradually increased (e.g. up to 16 SD above baseline).

### Results

Rats respond to the increasing SWR threshold by increasing the rate of occurrence of SWRs of all sizes, thereby increasing the chances of generating a large event that will trigger the detection criteria and lead to reward. After two weeks of conditioning, the SWR occurrence rate at the SWR port is approximately double the rate observed at the Control port, and this increase is stable throughout the time spent at both ports. Importantly, changes in SWRs were not seen at other points during the task or during subsequent sleep epochs, demonstrating that the conditioning is trial-phase specific. Further, no detectable differences between the SWRs were observed at either port; SWRs at both ports included decoded content representing the outer arms of the task, suggesting that the conditioned SWRs are physiologically normal events with task-relevant content. Critically, since the number of SWRs is greater at the SWR port, the total amount of arm representation during SWR trials on average is significantly more than during Control trials. Thus, providing reward feedback coupled to SWR detection, and challenging the subjects to generate larger SWRs, causes an increase in the occurrence rate of normal, task-relevant SWRs. Further studies will evaluate whether this paradigm can provide cognitive benefit to the subject.

### Implications

The efficacy of this training paradigm demonstrates that when provided with meaningful feedback in the context of a SWR-conducive behavioral state, rats are able to learn to modulate their own SWR rate. Given the substantial evidence connecting SWRs with memory processes, SWR rate modulation may influence or enhance memory ability. Unlike other SWR manipulation studies (Girardeau et al. (2009) Nat Neurosci., vol. 12, pp. 1222-1223; Ego-Stengel et al. (2010) Hippocampus, vol. 20, pp. 1-10; Jadhav et al. (2012) Science, vol. 336, pp. 1454-8; Fernandez-Ruiz et al. (2019) Science, vol. 364, pp. 1082-1086), this strategy of SWR modulation can be readily adapted for human use. SWRs in humans can be detected on standard depth electrodes placed in the hippocampus, and sensory feedback can be provided, triggered by SWRs, with low latency and minimal software. Training with this feedback could enable patients to learn mental strategies for increasing SWR occurrence, could become more adept at reaching this state, and potentially improve memory ability.

A SWR-based neural feedback training system of the present disclosure can be applied in the context of patients receiving medial temporal lobe grid and depth electrode arrays for the purpose of seizure monitoring for medically refractory epilepsy. During their extended stay in hospital for monitoring (approximately a week), subjects could receive sensory feedback triggered by the detection of SWRs during a range of behavioral tasks and quiet rest. The patient would be informed that patterns of neural activity related to memory processes were being detected and triggering the feedback, and that the patient should attend to and note mental state when such events occur. During quiet rest, previous work has measured an average (but variable) rate of 1.9 events/minute (Axmacher et al. (2008) Brain, vol. 131, pp. 1806-17). Such feedback could take various forms, all sharing the following general features: brief, positive, non-alarming or distracting, and unmistakeable. For instance, SWR detection could trigger a gold star icon appearing on a tablet screen accompanied by a chime noise. Such feedback could occur during quiet rest while the patient was instructed to simply let the mind wander and see if he/she could identify thought patterns or mental strategies that increased SWR occurrence. Alternatively, the patient could participate in a more structured task which would involve, in addition to short periods of quiet rest, several minute long blocks of diverse memory- related activities. For instance, these might include activities/prompts such as recounting a story from childhood (remote episodic memory), describing events earlier in the day (recent episodic memory), naming state capitols (semantic memory), describing a route of their daily commute (spatial memory), and describing steps of a familiar process (sequential non-spatial memory). Non-memory-dependent prompts would also be interspersed, such as reading a sentence aloud, tracing a simple drawing, or doing basic arithmetic problems. SWR-triggered feedback would be provided throughout. The memory-engaging prompts would be expected to cause increase SWR occurrence, while less would be expected during non-memory-engaged tasks. The structured progression through prompts with varying memory engagement could promote exploration and engagement or various mental states, allowing the patient to experience and notice trends in mental state conducive to SWRs. With training, the subject could become more adept at engaging a SWR-conducive state and generating more SWRs.

Based on the understanding of the role of SWRs in memory processes, there is potential for such training to cause a general increase in subject's ability to retrieve memories of previously stored facts and events. This could manifest as faster learning and potentially improved memory-guided decision-making, etc. These effects could be measured in a small time frame; ie, in the same day as the SWR training or throughout the week of seizure monitoring. Alternatively, if a reliably pro-SWR mental state could be achieved, the long-term (on the order of years) effects of regularly employing pro-SWR mental strategies could be measured.

As the ability to record SWRs with less invasive measures improves, SWR-based training will become more accessible. Advances are already being made in detecting replay events using MEG, which is non-invasive although not portable or convenient. However, initial studies could use such a MEG technique to assess efficacy of the ripple training in patients suffering from memory loss, such as Alzheimer' s patients, to see whether cognitive decline could be slowed or reversed by increased SWR occurrence. Implanted recording devices similar to a deep brain stimulation probe could be implanted solely for the purpose of SWR-based training. However, noninvasive recording techniques will also be able to detect SWRs, making SWR training dependent on a potentially small wearable detector device and for example, a phone app to provide feedback. Such an implementation could make SWR-based training extremely low-barrier, accessible, and maintainable.

Overall, the key concept of linking external sensory feedback to internally generated patterns of neural activity supporting memory processes (SWRs) is a novel and potentially applicable strategy to preserve or even enhance memory function in humans.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of modulating hippocampal replay, the method comprising:
a) recording a plurality of signals from one or more regions of the brain of a subject;
b) filtering the plurality of signals to a frequency ranging from 20-250 Hz;
c) detecting sharp wave ripple (SWR) activity above a set threshold in one or more regions of the brain;
e) providing feedback to the subject triggered by the detection of SWR activity in the subject above a set threshold,
wherein said providing feedback to the subject modulates the SWR activity in the subject.

2. The method of claim 1, wherein said feedback increases the SWR activity in the subject.

3. The method of any one of claims 1-2, wherein said feedback increases SWR occurrence rate in the subject.

4. The method of any one of claims 1-3, wherein said feedback enhances memory performance in the subject.

5. The method of any one of claims 1-4, wherein said recording occurs when the subject is performing a memory-related or non-memory related behavioral task.

6. The method of any one of claims 1-5, wherein the method further comprises performing behavioral training on the subject during recording of the plurality of signals.

7. The method of any one of claims 1-6, wherein the subject is a non-human mammal.

8. The method of any one of claims 1-7, wherein the plurality of signals are neural signals.

9. The method of any one of claims 1-8, wherein the plurality of signals are MEGI signals, EEG signals, fMRI signals, or ECoG signals.

10. A method of enhancing or increasing memory performance in a subject, the method comprising:
a) recording a plurality of signals from one or more regions of the brain of a subject;
b) filtering the plurality of signals to a frequency ranging from 20-250 Hz;
c) detecting sharp wave ripple (SWR) activity above a set threshold from the filtered signals in the one or more regions of the brain;
e) providing feedback to the subject triggered by the detection of SWR activity in the subject above a set threshold,
wherein said providing feedback to the subject increases SWR activity in the subject, wherein an increase in in SWR activity in the subject enhances or increases memory performance in the subject.

11. The method of claim 10, wherein said recording is performed by an electrode array.

12. The method of any one of claims 10-11, wherein the method further comprises instructions that, when executed by one or more processors, cause the one or more processors to send sensory feedback to the subject.

13. The method of any one of claims 10-12, wherein said feedback is sent via tablet, computer, or phone.

14. The method of any one of claims 10-13, wherein the method is carried out using a receiver unit, comprising:
a receiver in communication with a transmitter that receives the plurality of signals detected from the at least three electrodes;
one or more processors;
a non-transient computer-readable medium comprising instructions that, when executed by the one or more processors, cause the one or more processors to:
perform one or more filters on the plurality of signals;
detect SWR activity based on the plurality of signals; and
provide feedback to the subject triggered by SWR activity.

15. A method of reducing or treating memory loss in a subject, the method comprising:
a) recording a plurality of signals from one or more regions of the brain of a subject;
b) filtering the plurality of signals to a frequency ranging from 20-250 Hz;
c) detecting sharp wave ripple (SWR) activity above a set threshold in the one or more regions of the brain;
e) providing feedback to the subject triggered by the detection of SWR activity in the subject above a set threshold,
wherein said providing feedback to the subject increases SWR activity in the subject, wherein an increase in in SWR activity in the subject enhances or increases memory performance in the subject, thereby reducing or treating memory loss in the subject.

16. The method of claim 15, wherein said recording is performed by an electrode array.

17. The method of any one of claims 15-16, wherein the method further comprises instructions that, when executed by one or more processors, cause the one or more processors to send sensory feedback to the subject.

18. The method of any one of claims 15-17, wherein said feedback is sent via tablet, computer, or phone.

19. The method of any one of claims 15-18, wherein the method is carried out using a receiver unit, comprising:
a receiver in communication with a transmitter that receives the plurality of signals detected from the at least three electrodes;
one or more processors;
a non-transient computer-readable medium comprising instructions that, when executed by the one or more processors, cause the one or more processors to:
apply one or more filters on the plurality of signals;
detect SWR activity based on the plurality of signals; and
provide feedback to the subject triggered by SWR activity.

20. A system that modulates hippocampal replay and/or SWR activity in a subject, the system comprising:
an electrode array in contact with one or more regions of the brain of the subject;
an electrical recording device configured to record a plurality of signals in the one or more regions of the brain;
one or more processors,
a non-transient computer-readable medium comprising instructions that, when executed by the processor, cause the processor to:
apply one or more filters on the plurality of signals to filter the plurality of signals to a frequency ranging from 20-250 Hz;
detect sharp wave ripple (SWR) activity in the subject from the filtered signals that exceed a set threshold;
a feedback source configured to provide feedback to the subject triggered by the detection of SWR activity, wherein the feedback to the subject is configured to modulate SWR activity in the subject.

21. The system of claim 20, wherein feedback from the feedback source is configured to increase SWR occurrence in the subject.

22. The system of any one of claims 20-21, wherein feedback from the feedback source is configured to increase SWR activity in the subject.

23. The system of any one of claims 20-22, wherein feedback from the feedback source is configured to increase the size of SWR in the subject.

24. The system of any one of claims 20-23, wherein the plurality of signals are recorded when the subject is performing a behavioral task or during quiet rest.

25. The method of claim 24, wherein the behavioral task includes identifying thought patterns and mental strategies that increase SWR occurrence.

26. The system of claim 24, wherein the behavioral task comprises at least one of: short periods of quiet rest; and blocks of diverse memory-related and/or non memory-related activities for a period of time.

27. The system of claim 26, wherein the memory-related activity comprises one or more of:
recounting a story from childhood, describing events earlier in the day, naming state capitols, describing a route of their daily commute, and describing steps of a familiar process.

28. The system of claim 24, wherein the behavioral task comprises non-memory-dependent prompts.

29. The system of claim 26, wherein the non-memory dependent prompts comprise reading a sentence aloud, tracing a drawing, or performing arithmetic problems.

30. The system of claim 27, wherein the memory-related activity is configured to cause an increase in SWR occurrence as compared to the same subject prior to training

31. The system of any one of claims 20-30, wherein the plurality of signals are neural signals.

32. The system of any one of claims 20-31, wherein the plurality of signals are recorded by a Magnetoencephalographic Imaging (MEGI), an Electroencephalography (EEG) device, functional magnetic resonance imaging (fMRI) device, or a Electrocorticography (ECoG) device.

33. The system of any one of claims 20-32, wherein the plurality of signals are filtered to a frequency ranging from 30-50 Hz.

34. The system of any one of claims 20-32, wherein the plurality of signals are filtered to a frequency ranging from 50-80 Hz.

35. The system of any one of claims 20-32, wherein the plurality of signals are filtered to a frequency ranging from 80-150 Hz.

36. The system of any one of claims 20-32, wherein the plurality of signals are filtered to a frequency ranging from 150-250 Hz.

37. The system of any one of claims 20-36, wherein the region of the brain is the hippocampus.

38. The system of any one of claims 20-37, wherein the array of electrodes comprise an array of tetrodes.

39. The system of any one of claims 20-38, wherein the feedback source comprises a sensory feedback from the feedback source.

40. The system of any one of claims 20-39, wherein the feedback is sent from the feedback source comprising a tablet, computer, or phone.

41. The system of any one of claims 20-41, wherein the subject has a disease or disorder selected from the group consisting of: dementia, Alzheimer's disease, memory loss, and epilepsy.

42. The system of any one of claims 20-41, wherein the non-transient computer-readable medium comprises instructions that, when executed by the processor, cause the processor to increase the threshold for detection of SWR activity of the subject.

43. A system for enhancing memory performance in a subject, the system comprising:
an electrode array in contact with one or more regions of the brain of the subject;
an electrical recording device configured to record a plurality of signals in the one or more regions of the brain;
one or more processors,
a non-transient computer-readable medium comprising instructions that, when executed by the processor, cause the processor to:
perform one or more filters on the plurality of signals to filter the plurality of signals to a frequency ranging from 20-250 Hz;
detect sharp wave ripple (SWR) activity in the subject from the filtered signals that exceed a set threshold;
a feedback source configured to provide feedback to the subject triggered by the detection of SWR activity, wherein the feedback to the subject is configured to increase SWR activity in the subject as compared to a subject without said feedback, wherein the increase in SWR activity results in enhanced or increased memory performance
